(19) United States
(12) Patent Application Publication
(10) Pub. No.: US 2007/0269797 A9
(43) Pub. Date: Nov. 22, 2007

(54) Method for analysis of the phenotypic characteristics of the human immunodeficiency virus (HIV)

(75) Inventors: Francois Clavel, Paris (FR); Fabrizio Mammano, Paris (FR); Esther Race, Thame (GB); Elisabeth Dam, Paris (FR); Veronique Obry, La Garenne-Colombes (FR); Virginie Trouplin, Paris (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: BioAlliance Pharma S.A. and Inserm

(21) Appl. No.: 10/435,659

(22) Filed: May 12, 2003

Related U.S. Application Data
(63) Continuation of application No. PCT/FR01/03512, filed on Nov. 9, 2001.
Application No. PCT/FR01/03512 is a continuation-in-part of application No. 09/817,135, filed on Mar. 27, 2001, now abandoned.

(30) Foreign Application Priority Data
Nov. 10, 2000 (FR) 00/14495
Mar. 23, 2001 (FR) 01/03970

(51) Int. Cl. C12Q 1/70

(52) U.S. Cl. 435/5

(57) ABSTRACT

The present invention relates to a method for analyzing the phenotypic characteristics shown by certain virus strains, particularly human immunodeficiency viruses, involving the construction of a recombinant virus obtained by homologous recombination. The present invention also relates a kit comprising the primers, vectors, cell hosts, products and reagents required to carry out PCR amplification, and the products and reagents used to detect a marker, for the implementation of the method according to the invention.

### FIELD OF THE INVENTION

This invention relates to a method for analysis of the phenotypic characteristics that certain virus stocks, in particular the HIV virus, exhibit.

### BACKGROUND OF THE INVENTION

Genotypic tests that are quick and widely available for detecting the presence of mutations in viral genes have been developed, for example, for detecting mutations that are present in the genes that code for the protease or the reverse transcriptase of HIV.

Although certain mutations have been associated with a particular inhibitor of viral activity, others are associated with treatment by several molecules. In addition, with the development of new inhibitor molecules, the genotyping of virus variants that defy treatment becomes increasingly complex. This makes difficult the evaluation and the fact of knowing for which inhibitors the viruses become resistant or still preserve a slight susceptibility. Under these circumstances, the presence and the accumulation of resistance mutations can surely constitute a good index of the evolution of the resistance, but the simple detection of these mutations on the genotypic level is no longer adequate for evaluating quantitatively the resistance level, a parameter that proves crucial for optimizing the therapeutic orientations of patients with whom antiviral therapy fails.

The phenotypic characteristics of the viruses are linked to various aspects of viral behavior and are directly involved in numerous interactions between said viruses and their environment.

Only the phenotypic tests, which measure directly in the culture medium the modification of the phenotypic characteristic of the virus, such as the inhibition of the viral activity, for example in the presence of inhibitor compounds of said viral activity, provide a quantitative index of the resistance.

Other phenotypic characteristics of which analysis proves interesting, in particular from a medical viewpoint, are those that impart to a virus its resistance regarding inhibitor agents that can block at least one mechanism that is involved in the viral activity, those that impart to a virus its replicative capacity, those that impart to a virus its tropism to particular targets or else those that impart to a virus its aptitude to be neutralized by molecules, such as antibodies, chemokines or inhibitors.

This invention makes it possible to test several phenotypic characteristics that are distinct but complementary to HIV with a quick method that is based on the reconstruction of a recombinant virus starting from samples taken from infected patients and involving a unique cycle of viral replication. This invention is part of a process for decision-making and optimization of therapeutic strategy in HIV-infected patients for whom antiretroviral treatment has failed virologically or else in patients who have not undergone antiretroviral treatment. Only a complete knowledge of the different phenotypic characteristics of the virus can effectively help the clinician to make therapeutic decisions that are best suited to the particular situation of his patient.

Among the phenotypic characteristics of the HIV virus whose analysis exhibits a particular advantage from the medial viewpoint are found those that are linked to the expression of genes that can undergo at least one mutation, located in the GAG, ENV or POL regions of the viral genome, such as those listed below:

I Infectivity, Replicative Capacity and Virulence

These phenotypic characteristics of the HIV virus can be linked to the function of all of the viral genome regions, in the case of parts that code for proteins or regions that occur in different mechanisms or stages of the viral replication cycle. In particular, it is important to evaluate the effect that is produced by the mutations in genes that code for protease, the reverse transcriptase, the integrase or the envelope on viral replication and most particularly with viruses that have developed a resistance to antiviral agents.

Its analysis makes it possible to measure the replicative capacity of a virus, also called infectivity or “fitness.”

II Susceptibility/Resistance to Reverse Transcriptase Inhibitors

This phenotypic characteristic of the HIV viruses is linked to the expression of a portion of the POL region that codes for the reverse transcriptase.

Its analysis allows the adjustment of antiretroviral treatments by nucleosidic analogs or non-nucleosidic reverse transcriptase inhibitors (NNRTI).

III Susceptibility/Resistance to Integrase Inhibitors

This phenotypic characteristic of the HIV viruses is linked to the expression of a portion of the POL region that codes for the integrase.

Its analysis provides indications for adjusting antiretroviral treatments by integrase inhibitors.

IV Susceptibility/Resistance to Inhibitors of Virus Entry into the Target Cell

This phenotypic characteristic of the HIV viruses is linked to the expression of the glycoprotein of the HIV envelope and in particular to the expression of the transmembrane subunit of said glycoprotein that is coded by a part of the ENV gene.

The analysis of this phenotypic characteristic allows the demonstration of the action of inhibitor agents that block the fusion of the viral membrane with the membrane of the target cell.

V Susceptibility/Resistance to Inhibitors that Target the Co-Receptors of the HIV Viruses

This phenotypic characteristic is linked also to the expression of at least a portion of the ENV region of the HIV viruses that codes for polypeptides that participate in the connection with the co-receptors of the target cell. The envelope/co-receptor interaction allows the HIV virus to enter said target cell.

Its analysis makes it possible to measure the resistance of the HIV viruses to the action of inhibitors that block the co-receptors that are used by the HIV to carry out its entry into the target cell.

The inhibitor agents interfere with the co-receptor by inhibiting its interaction with the HIV envelope.

In particular, it is important to evaluate the effect of mutations in the ENV region that modify the interaction of certain regions of the envelope protein with the CXCR4 or CCR5 receptors of the HIV target cells.

VI Tropism

This phenotypic characteristic is also linked to the expression of at least a portion of the ENV region that codes for polypeptides of the envelope of HIV viruses that participate in the connection with one or more receptors of the target cell, in particular with the CXCR4 or CCR5 co-receptors.

Its analysis makes it possible to measure in vivo the capacity of the HIV virus to use said receptors, in particular CXCR4 or CCR5, which are expressed in a different way in various cellular types and makes it possible to know if the virus uses one or the other of the receptors, or both.

The indications provided by this analysis make it possible to deduce the viral behavior in some types of cells, natural targets of the HIV.

VII Virulence

This phenotypic characteristic is also linked to the expression of all or part of the ENV region that codes for the envelope peptides of the HIV virus.

Its analysis makes it possible to evaluate the cytopathogenic power of an HIV virus.

VIII Neutralizing Capacity

This phenotypic characteristic is also linked to the expression of envelope proteins of the HIV viruses.

Its analysis makes it possible to evaluate the susceptibility of viruses to the inhibiting action of antibodies or substances that are naturally present in the organism and present in the serum or in other fluids.

The first tests for detecting, for example, the phenotypic characteristic of resistance of HIV viruses to antiviral treatments were carried out by using primary isolates and peripheral blood lymphocytes (PBL) stimulated by phytoagglutinin (PHA) according to a procedure that is laborious and difficult to reproduce. An innovative alternative to these tests, a recombinant virus test, named RVA below, was proposed by Kellam and Larder in 1994.

This RVA analytical method measured the resistance of a recombinant virus carrying the reverse transcriptase isolated from the plasma of a virus-carrying patient by co-transfection of the sequences of the latter duly amplified by a polymerization chain reaction (PCR) with a virus clone obtained in a laboratory, which is deleted from its reverse transcriptase and is competent for replication in a variety of well-plotted cellular lines.

Several modifications of this method have now been described (Boucher, C.; Keulen, W.; Bommel, T.; Nijhuis, M.; Jong, D.; Jong, M.; Schipper, P. and Back, N., K. (1996) “HIV-1 Drug Susceptibility Determination by Using Recombinant Viruses Generated from Patient Sera Tested in a Cell-Killing Assay. Antimicrobial Agents and Chemotherapy” 40 (10), 2404-2409) (Shi, C. and Mellors, J. W. (1997) “A Recombinant Retroviral System for Rapid In Vivo Analysis of Human Immunodeficiency Virus Type 1 Susceptibility to Reverse Transcriptase Inhibitors.” Antimicrob Agents Chemother 41 (12), 2781-5) (Hertogs, K.; de Bethune, M. P.; Miller, V.; Ivens, T.; Schel, P.; Van Cauwenberge, A.; Van Den Eynde, C.; Van Gerwen, V.; Azijn, H.; Van Houtte, M.; Peeters, F.; Staszewski, S.; Conant, M.; Bloor, S.; Kemp, S.; Larder, B. and Pauwels, R. (1998) “A Rapid Method for Simultaneous Detection of Phenotypic Resistance to Inhibitors of Protease and Reverse Transcriptase in Recombinant Human Immunodeficiency Virus Type 1 Isolates from Patients Treated with Antiretroviral Drugs.” Antimicrob Agents Chemother 42(2), 269-76.) (Hecht, F. M.; Grant, R. M.; Petropoulos, C. J.; Dillon, B.; Chesney, M. A.; Tian, H.; Hellmann, N. S.; Bandrapalli, N. I.; Digilio, L., Branson, B. and Kahn, J. O. (1998) “Sexual Transmission of an HIV-1 Variant Resistant to Multiple Reverse-Transcriptase and Protease Inhibitors.” N Engl J Med 339 (5), 307-11) (Medina, D. J.; Tung, P. P.; Nelson, C. J.; Sathya, B.; Casareale, D. and Strair, R. K. (1998) “Characterization and Use of a Recombinant Retroviral System for the Analysis of Drug Resistant HIV.” J Virol Methods 71 (2), 169-76).

Most of these recombinant systems, however, exhibit drawbacks because, as for the method that uses PBMC, the production of a reserve of infectious particles expressing a particular phenotypic characteristic that it is sought to detect and to measure requires an amplification of the virus by exponential growth of the lymphocytic cells. The virus is then subjected to genetic derivatives during its replication and can lose mutations that are essential for the expression of the desired phenotypic characteristic that thus modifies the reliability of the method.

Another drawback for indicating, for example, a phenotypic characteristic of resistance that is present in the methods for analysis of the prior art comes from the fact that the simultaneous presence of several mutations that can impart a resistance relative to different retroviral inhibitors reduces the replicative capacity of the virus.

### SUMMARY OF THE INVENTION

The inventors developed a new method for analysis of a phenotypic characteristic of the HIV viruses that requires only one replication cycle.

This method that requires only a single viral replication cycle is implemented by the selection of culture conditions of the recombinant viruses that are obtained.

These culture conditions relate to the monitoring of the culture time, selected for preventing the viral replication beyond the first cycle; this culture time is between 12 hours to 72 hours, preferably 24 hours to 48 hours.

The culture conditions also relate to the selection of cells of the first cellular system; they are selected so that they are not permissive with regard to viral infection, for example such cells do not have the CD4 receptor that is necessary to the entry of the HIV virus into the cell. Examples of such cells are the HeLa or 293T cells.

Finally, these culture conditions also relate to the recombinant viruses that are constructed according to the method of the invention, which exhibit an envelope protein deficit. These viruses, once produced in the first cellular system, are actually incapable of reinfecting the cells of this first cellular system.

This analytical method is based on the construction of a recombinant virus (RAV) that is obtained by cotransfection and recombination homologous with:
a) The DNA sequences that are obtained from an HIV to be analyzed and that can comprise mutations that can modify the desired phenotypic characteristic, whereby said sequences are extracted from a biological medium such as plasma, serum, saliva, sperm or other secretions, from a patient who is a carrier of said virus,
b) A first vector that exhibits a specific deletion of sequences that allow the replication of the HIV as well as a deletion of all or part of the sequence that imparts to the HIV the desired phenotypic characteristic and
c) A second vector, for example, a plasmid, that comprises the sequences that complete those that are necessary for the replication of said virus and that are absent from the first vector.

The method developed by the inventors is quick; it requires about seven days to be carried out and can then be used for routine determinations such as the measurement of the susceptibility of HIV-infected patients to inhibitors of viral activity.

Thus, in patent U.S. Pat. No. 6,103,462, the inventors already described a first application of this analytical method, based on the formation of a particular recombinant virus to determine the susceptibility of an HIV virus to protease inhibitors.

### DETAILED DESCRIPTION OF PREFERRED EMBODIMENTS

The new analytical methods that ate used within the scope of this HIV invention are based on the determination of HIV virus phenotypic characteristics that are associated with mutations that can be present at least in a gene that is selected from among the group that comprises the genes gag, pol, protease, reverse transcriptase, RNAse H, integrase, vif, vpr, tat, rev, vpu, env, nef, cis-active sequences, LTR, dimerization sequences, splicing-regulating sequences, and RRE with ad-hoc recombinant viruses.

The invention therefore relates to a method for analysis of a phenotypic characteristic of HIV viruses that are present in a biological sample from a patient, whereby said phenotypic characteristic results from one or more mutations of the viral genome that can influence the viral infection, characterized in that it comprises:
a) The extraction of nucleic acids that are contained in a biological sample,
b) At least one amplification by PCR of a segment of the nucleic acids of stage (a), each with a pair of primers that frame a nucleic acid sequence of the viral genome that can carry at least one mutation,
c) The preparation of a vector that comprises the parts of a genome of an HIV virus that are necessary for the viral replication except for the amplified segment in stage (b) and optionally with the exception of the gene that codes for the envelope protein,
d) The transfection of a first cellular host with:
   The nucleic acids that are obtained in stage (b),
   The vector that is prepared in stage (c),
   Optionally a second vector that comprises a gene that codes for an envelope protein if the envelope gene is deleted from the vector that is prepared in stage (c) to obtain a chimerical virus by homologous recombination,
e) The culture of said first cellular host under conditions that make it possible to produce vital particles during a single replication cycle,
f) The infection by the viral particles that are obtained in stage (e) from at least a second cellular host that can be infected by an HIV virus or an HIV-pseudotype virus and that optionally comprises a marker gene that can be activated only following viral infection, and
g) The detection and/or the quantification of the marker that is expressed in stage (f) so as to demonstrate at least one phenotypic characteristic of the HIV viruses that are present in the biological sample.

More particularly, the amplification by PCR of stage (b) is carried out with a pair of primers that frame a nucleic acid sequence that comprises all or part of a viral genome region that is selected from among: gag, pol, protease, reverse transcriptase, RNAse H, integrase, vif, vpr, tat, rev, vpu, env, nef, cis-active sequences, LTR, dimerization sequences, splicing-regulating sequences or the Rev response element (RRE).

According to a particular embodiment of the analytical method of the invention, the amplification by PCR of stage (b) is carried out with a pair of primers that frame a nucleic acid sequence that codes for a portion of the gag protein of the human immunodeficiency virus and a nucleic acid sequence that codes for the protease that can carry at least one mutation in the gene that codes for the protease and in that the vector of stage (c) is constructed from a genome of an HIV virus where all or part of the gene that codes for the protease is deleted.

Advantageously, the amplification of stage (b) according to the analytical method of the invention of a nucleic acid sequence that can carry at least one mutation in the gene that codes for the protease is made with a pair of primers having a size of between 10 and 50 oligonucleotides, comprising the sequences Fit A−: (5′ TCA CCT AGA ACT TTA AAT GC 3′) (SEQ ID No: 1) and Pro A−: (5′ GGC AAA TAC TGG AGT ATT GTA TG3′ 3′) (SEQ ID No: 2), or constituted of fragments of the latter or else sequences analogous to the latter that carry mutations of one or more nucleotides that do not significantly modify their capacity to hybridize the region of the gene of the protease that carries the mutation or mutations, followed by a second amplification with a pair of primers having a size of between 10 and 50 oligonucleotides, comprising the sequences: Fit B: (5′ AGA ACT TTA AAT GCA TGG GT 3′) (SEQ ID No: 3) and Pro B−: (5′ GGA GTA TTG TAT GGA TTT TCA GG 3′) (SEQ ID No: 4), or constituted of fragments of the latter, or else sequences analogous to the latter that carry mutations of one or more nucleotides that do not significantly modify their capacity to hybridize the region of the gene of the protease that carries the mutation or mutations.

Quite preferably, the amplification of stage (b) according to the analytical method of the invention of a nucleic acid sequence that can carry at least one mutation in the gene that codes for the protease is made with a pair of primers:
Fit A−: (5′ TCA CCT AGA ACT TTA AAT GC 3′) (SEQ ID No: 1) and
Pro A−: (5′ GGC AAA TAC TGG AGT ATT GTA TG3′ 3) (SEQ ID No: 2), followed by a second amplification with a pair of primers:
   Fit B: (5′ AGA ACT TTA AAT GCA TGG GT 3′) (SEQ ID No: 3), and
   Pro B−: (5′ GGA GTA TTG TAT GGA TTT TCA GG 3′) (SEQ ID No: 4),
to obtain a DNA segment of 1488 base pairs that extend between residues 1237 and 2725 inclusive, and the vector of stage (c) is a retroviral vector that is deleted from the region of the pol reading frame that codes for the HIV-1 protease that extends from residues 1505 to 2565 inclusive, deleted from the envelope region and comprising a single restriction site MluI.

According to a second particular embodiment of the analytical method of the invention, the amplification by PCR of stage (b) of the analytical method according to the invention is carried out with a pair of primers that frame a nucleic acid sequence that can carry at least one mutation in the gene that codes for the reverse transcriptase, and the transfection of stage (c) is carried out with a first vector that is constructed from a genome of an HIV virus where all or part of the gene that codes for the reverse transcriptase is deleted.

Advantageously, the amplification of stage (b) according to the analytical method of the invention, with a pair of primers that frame a nucleic acid sequence that can carry at least one mutation in the gene that codes for the reverse transcriptase, is made with a pair of primers having a size of between 10 and 50 oligonucleotides, comprising sequences MJ3 (5′ AGT AGG ACC TAC ACC TGT CA 3′) (SEQ ID No: 5) and RT-EXT (5′ TTC CCA ATG CAT ATT GTG AG 3′) (SEQ ID No: 6), or constituted of fragments of the latter, or else sequences analogous to the latter that carry mutations of one or more nucleotides that do not significantly modify their capacity to hybridize the region of the gene of the transcriptase that carries at least one mutation, followed by a second amplification stage with a pair of primers comprising the sequences: A35 (5′ TTG GTT GCA TAA ATT TTC CCA TTA GTC CTA TT 3′) (SEQ ID No: 7) and RT-IN (5′ TTC CCA ATG CAT ATT GTG AG 3′) (SEQ ID No: 8) or constituted of fragments of the latter, or else sequences that are analogous to the latter that carry mutations of one or more nucleotides that do not significantly modify their capacity to hybridize the region of the gene of the reverse transcriptase that carries at least one mutation.

Quite preferably, the amplification of stage (b) according to the analytical method of the invention, with a pair of primers that frame a nucleic acid sequence that can carry at least one mutation in the gene that codes for the reverse transcriptase is made with a pair of primers:
MJ3 (5′ AGT AGG ACC TAC ACC TGT CA 3′) (SEQ ID No: 5) and
RT-EXT (5′ TTC CCA ATG CAT ATT GTG AG 3) (SEQ ID No: 6), followed by a second amplification stage with a pair of primers:
   A35 (5′ TTG GTT GCA TAA ATT TTC CCA TTA GTC CTA TT 3′) (SEQ ID No: 7) and
   RT-IN (5′ TTC CCA ATG CAT ATT GTG AG 3′) (SEQ ID No: 8)
to obtain a DNA segment of 1530 base pairs that extend beyond codon 93 of the region that codes for the protease and beyond codon 503 of the region that codes for the polymerase (POL), and the vector of stage (c) is a retroviral vector that is deleted from the region of the pol reading frame that codes for the HIV-1 reverse transcriptase that extends from residues 2618 to 2872 inclusive and comprises a unique restriction site MluI.

According to a third embodiment, the invention also has as its object an analytical method according to which the amplification of stage (b) is carried out with a pair of primers that frame a nucleic acid sequence that codes for a portion of the gag protein, for the protease and for a portion of the reverse transcriptase of the human immunodeficiency virus that can carry at least one mutation in the nucleic acid sequence that codes for the gag protein or for the protease or for the reverse transcriptase.

Quite preferably, the amplification of stage (b) with a pair of primers that frame a nucleic acid sequence that codes for a portion of the gag protein, for the protease and for a portion of the reverse transcriptase of the human immunodeficiency virus that can carry at least one mutation in the nucleic acid sequence that codes for the gag protein or for the protease or for the reverse transcriptase is carried out with the pair of primers:
gag+1 (5′ AGGGGCAAATGGTACATCA 3′) (SEQ ID No: 31) and
RT-EX (SEQ ID No 6),
followed by a second amplification stage with a pair of primers:
   Fit B+ (SEQ ID No: 1) and
   RT-IN (SEQ ID No: 8)
to obtain a DNA segment of 2825 base pairs that extend between residues 1237 and 4062, and the transfection of stage (c) is carried out with a retroviral vector that is deleted from a portion of the gag gene and regions within the pol reading frame that codes for the protease and a portion of the HIV-1 reverse transcriptase that extends from residues 1507 to 3870 inclusive, deleted in the envelope region and comprising a unique restriction site NruI.

Advantageously, the virus that results from the transfection that is mentioned above can be used to determine the infective or replicative capacity of the virus that has mutations in the reverse transcriptase and/or protease. The quantification of the viral particles according to this last analytical method is accomplished by the measurement of the antigen p24. It should be pointed out that a similar analytical method can in no case be carried out by using a vector that is deleted simply from the sequence of the reverse transcriptase since these non-viable recombinant viruses can also produce antigen p24. By constructing a recombinant virus that has an additional deletion of the region corresponding to the protease, only the correctly recombined viruses produce the antigen p24 and thus make possible a certain and reliable measurement of the viral particles that are produced.

The invention also relates to an analytical method for determining the susceptibility of an HIV virus to an inhibiter compound of the reverse transcriptase, consisting in adding or not adding said inhibitor compound of the reverse transcriptase, optionally at different concentrations, to the second cellular host, prior to the infection of the latter by viral particles that are obtained in stage (a) and comprising in stage (g) the comparison of the expression of the marker gene with and without an inhibitor compound of the reverse transcriptase.

According to a third particular implementation of the analytical method of the invention, the amplification by PCR of stage (b) is carried out with a pair of primers that frame a nucleic acid sequence that can carry at least one mutation in the gene that codes for the integrase, and the vector of stage (c) is a retroviral vector that is deleted from all or part of the gene that codes for the integrase.

Advantageously, the amplification of stage (b) according to the analytical method of the invention with a pair of primers that frame a nucleic acid sequence that can carry at least one mutation in the gene that codes for the integrase is made with the pair of primers having a size of between 10 and 50 oligonucleotides, comprising the sequences: INT B+-5′ GTTACTAATAGAGGAAGACAAA3′ (SEQ ID No: 9) and INT B-5′ TTTTGGTGTTATTAATGCT3′ (SEQ ID No: 10), or else sequences analogous to the latter that carry mutations of one or more nucleotides that do not significantly modify their capacity to hybridize the region of the gene of the integrase carrying at least one mutation, followed by a second amplification stage, with the pair of primers: INT V+ 5′ CACCCTAACTGACACAACAA3′ (SEQ ID No: 11) and INT V− 5′ AAGGCCTTTCTTATAGCAGA3′ (SEQ ID No:12), or else sequences that are analogous to the latter and that carry mutations of one or more nucleotides that do not significantly modify their capacity to hybridize the region of the gene of the integrase that carries at least one mutation.

Quite preferably, the amplification of stage (b) according to the analytical method of the invention with a pair of primers that frame a nucleic acid sequence that can carry at least one mutation in the gene that codes for the integrase is made with the pair of primers:

| | |
|---|---|
| INT B+ | |
| −5′ GTTACTAATAGAGGAAGACAAA3′ and | (SEQ ID No: 9) |
| INT B− | |
| 5′ TTTTGGTGTTATTAATGCT3′, | (SEQ ID No: 10) |

followed by a second amplification stage, with the pair of primers:

| | |
|---|---|
| INT V+ | |
| 5′ CACCCTAACTGACACAACAA3′, and | (SEQ ID No. 11) |
| INT V− | |
| 5′ AAGGCCTTTCTTATAGCAGA3′, | (SEQ ID No. 12) |

to obtain a DNA fragment of 1460 base pairs that extend from residues 3950 to 5410 inclusive, and the vector of stage (c) is a retroviral vector that is deleted from the entire region of the pol reading frame that codes for the HIV-1 integrase that extends from residues 4228 to 5093 inclusive and from the region that codes for the viral envelope between positions 6343 and 7611 inclusive.

The invention also relates to an analytical method for determining the susceptibility of an HIV virus to an inhibitor compound of the integrase, consisting in adding or not adding said inhibitor compound of the integrase, optionally at different concentrations, during stage (e), before stage (f) and comprising in stage (g) the comparison of the expression of the marker gene with and without an inhibitor compound of the integrase.

According to a fourth particular implementation of the analytical method of the invention, the amplification by PCR of stage (b) is carried out with a pair of primers that frame a nucleic acid sequence that can carry at least one mutation in the gene that codes for the envelope protein, and the vector of stage (c) is a retroviral vector that is constructed from a genome of an HIV virus where all or part of the gene that codes for the envelope protein is deleted.

The vector of stage (c) is preferably a retroviral vector that is deleted from the entire region that codes for the extracellular portion of sub-unit gp41 of the HIV-1 envelope that extends from residues 7745 to 8263 inclusive, from the region of the HIV-1 genome that constitutes the Rev response element (RRE).

Advantageously, the amplification of stage (b) with a pair of primers that frame a nucleic acid sequence that can carry at least one mutation in the gene that codes for the envelope protein is made with a pair of primers having a size of between 10 and 50 oligonucleotides, comprising either sequences: FIN-A: 5′

| | |
|---|---|
| FIN-A: | |
| 5′TCAAATATTACAGGGCTGCT3′ | (SEQ ID No: 13) |
| and FIN-B: | |
| 5′TAGCTGAAGAGGCACAGG3′ | (SEQ ID No: 14) |
| or sequences FuA: | |
| 5′AAGCAATGTATGCCCCTCCCAT3′ | (SEQ ID No: 23) |
| and FuB: | |
| 5′GGTGGTAGCTGAAGAGGCACAGG3′ | (SEQ ID No: 24) |

GGTGGTAGCTGAAGAGGCACAGG3′ (SEQ ID No: 24) or constituted of fragments of the latter or else by sequences that are analogous to the latter carrying mutations of one or more nucleotides that do not significantly modify their capacity to hybridize the region of the gene of the envelope that carries at least one mutation followed by a second amplification stage, carried out with a pair of primers having a size of between 10 and 50 oligonucleotides, comprising either the sequences: FIN-C: 5′ CTATTAACAAGAGATGGTGG3′ (SEQ ID No: 15) and FIN-D: 5′ TCCACCTTCTTCTTCGATT3′ (SEQ ID No: 16), or sequences FuC′: 5′ ATATGAGGGACAATTGGAGAAGTGA3′ (SEQ ID No: 25), used in combination with a mixture of the following two sequences: FuD1: 5′ TCTGTCTCTCTCTCCACCTTCTTCTT3′ (SEQ ID No: 26) and FuD2: 5′ TCTGTCTTGCTCTCCACCTTCTTCTT3′ (SEQ ID No: 27) or constituted of fragments of the latter or else by sequences that are analogous to the latter that carry mutations of one or more nucleotides that do not significantly modify their capacity to hybridize the region of the gene of the envelope that carries at least one mutation.

Quite preferably, the amplification of stage (b) with a pair of primers that frame a nucleic acid sequence that can carry at least one mutation in the gene that codes for the envelope protein is made with a pair of primers:
FIN-A: 5′ TCAAATATTACAGGGCTGCT3′ (SEQ ID No: 13) and
FIN-B: 5′ TAGCTGAAGAGGCACAGG3′ (SEQ ID No: 14) followed by a second amplification stage that is carried out with the pair of primers:
   FIN-C: 5′ CTATTAACAAGAGATGGTGG3′ (SEQ ID No: 15) and
   FIN-D: 5′ TCCACCTTCTTCTTCGATT3′ (SEQ ID No: 16),
to obtain a DNA segment of 965 base pairs that extend from residues 7553 to 8517 inclusive and the vector of stage (c) is a retroviral vector that is deleted from the entire region that codes for the extracellular portion of sub-unit gp41 of the HIV-1 envelope that extends from residues 7745 to 8263 inclusive and comprises a unique restriction site Mull.

Quite preferably, the analytical method according to the invention makes possible the amplification of sequences of the envelope region of the HIV virus regardless of their sub-type and in particular viruses of sub-types A, B, C, D and E), by using, for the amplification stage (b), with a pair of primers that frame a nucleic acid sequence that can carry at least one mutation in the gene that codes for the envelope protein is made with a pair of primers:
FuA: 5′ AAGCAATGTATGCCCCTCCCAT3′ (SEQ ID No: 23) and
FuB: 5′ GGTGGTAGCTGAAGAGGCACAGG3′ (SEQ ID No: 24) followed by a second amplification stage that is carried out with the primer:
   FuC: 5′ ATATGAGGGACAATTGGAGAAGTGA3′ (SEQ ID No: 25) and a mixture of the following primers:
      FuD1: 5′ TCTGTCTCTCTCTCCACCTTCTTCTT3′ (SEQ ID No: 26)
      FuD2: 5′ TCTGTCTTGCTCTCCACCTTCTTCTT3′ (SEQ ID No: 27),
whereby said mixture is preferably carried out in a ratio of between (10%: 90%) and (90%: 100%) and quite preferably between (60%: 40%) and (40%: 60%),
to obtain a DNA segment of 805 base pairs that extend from residues 7635 to 8440 inclusive, and the vector of stage (c) is a retroviral vector that is deleted from the entire region that codes for the extracellular portion of sub-unit gp41 of the HIV-1 envelope that extends from residues 7745 to 8263 inclusive and comprises a unique restriction site Mu11.

Advantageously, the amplification of stage (b) with a pair of primers that frame a nucleic acid sequence that can carry at least one mutation in the gene that codes for the envelope protein is made with a pair of primers having a size of between 10 and 50 oligonucleotides, comprising the sequences: NEU-A: 5′ TAGAAAGAGCAGAAGACAGTGGCAATG3′ (SEQ ID No: 17) and FIN-B: 5′ TAGCTGAAGAGGCACAGG3′ (SEQ ID No: 14) or FuB: 5′ GGTGGTAGCTGAAGAGGCACAGG3′ (SEQ ID No: 24) or constituted of fragments of the latter, or else by sequences that are analogous to the latter that carry mutations of one or more nucleotides that do not significantly modify their capacity to hybridize the region of the gene of the envelope that carries at least one mutation, followed by a second amplification stage, with the pair of primers having a size of between 10 and 50 oligonucleotides, comprising the sequences: NEU-C: 5′ GTGGGTCACAGTCTATTATGGGG3′ (SEQ ID No: 18) and FIN-D: 5′ TCCACCTTCTTCTTCGATT3′ (SEQ ID No: 16) or a mixture of the following sequences: FuD1: 5′ TCTGTCTCTCTCTCCACCTTCTTCTT3′ (SEQ ID No: 26) and FuD2: 5′ TCTGTCTTGCTCTCCACCTTCTTCTT3′ (SEQ ID No: 27), or constituted of fragments of the latter, or else by sequences that are analogous to the latter carrying mutations of one or more nucleotides that do not significantly modify their capacity to hybridize the region of the gene of the envelope that carries at least one mutation.

Quite preferably, the amplification of stage (b) with a pair of primers that frame a nucleic acid sequence that can carry at least one mutation in the gene that codes for the envelope protein is made with a pair of primers:
NEU-A: 5′ TAGAAAGAGCAGAAGACAGTGGCAATG3′ (SEQ ID No: 17) and
FIN-B: 5′ TAGCTGAAGAGGCACAGG3′ (SEQ ID No: 14), followed by a second amplification stage, with the pair of primers:
   NEU-C: 5′ GTGGGTCACAGTCTATTATGGGG3′ (SEQ ID No: 18) and
   FIN-D: 5′ TCCACCTTCTTCTTCGATT3′ (SEQ ID No: 16),
to obtain a DNA fragment of between 2106 and 2320 base pairs that extend from residues 6197-6222 up to residues 6197-6222 inclusive and the vector of stage (c) is a retroviral vector that is deleted from the entire region that codes for the majority of sub-unit gp120 and the extracellular portion of gp41 of the HIV-1 envelope that extends from residues 6480 to 8263 inclusive and comprises a unique restriction site Mu11.

Quite preferably, the amplification of stage (b) with a pair of primers that frame a nucleic acid sequence that can carry at least one mutation in the gene that codes for the envelope protein is made with a pair of primers:
NEU-A: 5′ TAGAAAGAGCAGAAGACAGTGGCAATG3′ (SEQ ID No: 17) and
FuB: 5′ GGTGGTAGCTGAAGAGGCACAGG3′ (SEQ ID No: 24), followed by a second amplification stage, with the primers:
   NEU-C: 5′ GTGGGTCACAGTCTATTATGGGG3′ (SEQ ID No: 18) and a mixture of the following primers
      FuD1: 5′ TCTGTCTCTCTCTCCACCTTCTTCTT3′ (SEQ ID No: 26) and
      FuD2: 5′ TCTGTCTTGCTCTCCACCTTCTTCTT3′ (SEQ ID No: 27),
whereby said mixture is preferably carried out in a ratio that is between (100%:90%) and (90%:100%) and quite preferably between (60%:40%) and (40%:60%),
to obtain a DNA fragment of 2118 base pairs that extend from residues 6322 to 8440 inclusive, and the vector of stage (c) is a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mu11.

Advantageously, the amplification of stage (b) with a pair of primers that frame a nucleic acid sequence that can carry at least one mutation in the gene that codes for the envelope protein is made with a pair of primers having a size of between 10 and 50 nucleotides, comprising the sequences: E 00: 5′ TAGAAAGAGCAGAAGACAG-TCGCAATGA3′ (SEQ ID No: 19) and ES8B: 5′ CACTTCTCCAATTGTCCCTCA3′ (SEQ ID No: 20), or constituted of fragments of the latter, or else by sequences that are analogous to the latter that carry mutations of one or more nucleotides that do not significantly modify their capacity to hybridize the region of the gene of the envelope that carries at least one mutation, followed by a second amplification stage, with a pair of primers having a size of between 10 and 50 oligonucleotides comprising the sequences: E20: 5′ GGGCCACACATGCCTGTGTACCCACAG3′ (SEQ ID No: 21) and E115: 5′ AGAAAAATTCCCCTCCACAATTAA3′ (SEQ ID No: 22) or else by sequences that are analogous to the latter carrying mutations of one or more nucleotides that do not significantly modify their capacity to hybridize the region of the gene of the protease that carries at least one mutation.

Quite preferably, the amplification of stage (b) with a pair of primers that frame a nucleic acid sequence that can carry at least one mutation in the gene that codes for the envelope protein is made with a pair of primers:
E00: 5′ TAGAAAGAGCAGAAGACAGTGGCAATGA3′ (SEQ ID No: 19) and
ES8B: 5′CACTTCTCCAATTGTCCCTCA3′ (SEQ ID No: 20),
followed by a second amplification stage with the pair of primers:
   E20: 5′ GGGCCACACATGCCTGTGTACCCACAC3′ (SEQ ID No: 21) and
   E115: 5′ AGAAAAATTCCCCTCCACAATTAA3′ (SEQ ID No: 22),
to obtain a DNA segment of 938 base pairs that extend from residues 6426 to 7364 inclusive, and the vector of stage (c) is a retroviral vector that is deleted from the region, coding for the domains that extend from loop VI to loop V3 of the HIV-1 envelope that extends from 6617 to 7250 inclusive and comprises a unique restriction site NheI.

The invention also relates to an analytical method for determining the susceptibility of an HIV virus to a fusion-inhibitor compound that targets the gp41 protein of HIV-1, consisting in carrying out the amplification of stage (b) either with the pair of primers SEQ ID No: 13 and SEQ ID No: 14 followed by a second amplification with the pair of primers SEQ ID No: 15 and SEQ ID No: 16, or with the pair of primers SEQ ID No: 17 and SEQ ID No: 18, followed by a second amplification with the pair of primers SEQ ID No: 18 and SEQ ID No: 16, said fusion-inhibitor compound, optionally at different concentrations, to be added or not added during the cultivation of the cellular host that is obtained in stage (e), before stage (f) and comprising in stage (g) the comparison of the expression of the marker gene with and without a fusion-inhibitor compound targeting the gp41 of HIV-1.

The invention also relates to an analytical method for determining the susceptibility of an HIV virus to a compound that inhibits the entry of said HIV virus into a target cell, consisting in carrying out the amplification of stage (b) with the pair of primers SEQ ID No: 17 and SEQ ID No: 18 followed by a second amplification with the pair of primers SEQ ID No: 18 and SEQ ID No: 16, said compound that inhibits entry, optionally at different concentrations, to be added or not added to the cellular host that is obtained in stage (e) before the infection of stage (f) and comprising in stage (g) the comparison to the expression of the marker gene with and without a compound that inhibits entry.

The invention also relates to an analytical method for determining the susceptibility of an HIV virus to the inhibiting action of antibodies, consisting in carrying out the amplification of stage (b) with the pair of primers SEQ ID No: 17 and SEQ ID No: 18, followed by a second amplification with the pair of primers SEQ ID No: 18 and SEQ ID No: 16, said antibodies, optionally at different concentrations and comprising in stage (g) the comparison of the expression of the marker gene with and without antibodies, to be added or not added during cultivation stage (e).

The invention also relates to an analytical method for determining the tropism of an HIV virus for a cellular receptor, consisting in carrying out the amplification of stage (b) with the pair of primers SEQ ID No: 17 and SEQ ID No: 18, followed by a second amplification with the pair of primers SEQ ID No: 18 and SEQ ID No: 16, to carry out the infection of stage (f) with the viral particles that ate obtained in stage (e) on two separate cellular hosts and comprising in stage (g) the comparison of the expression of the marker gene by each of the two separate cellular hosts.

Advantageously, the cellular hosts that are used for the infection of stage (f) according to the analytical method of the invention are selected from among cellular hosts that express the CCR5 receptor or the CXCR4 receptor.

The invention also relates to an analytical method for determining the susceptibility of an HIV virus to an inhibitor compound that targets the co-receptors of HIV-1, consisting in carrying out the amplification of stage (b) with the pair of primers SEQ ID No: 17 and SEQ ID No: 18, followed by a second amplification with the pair of primers SEQ ID NO: 18 and SEQ ID No: 16, said inhibitor compound that targets the co-receptors of HIV-1, optionally at different concentrations, to be added or not added during cultivation stage (e), whereby the infection of stage (f) is carried out on two separate cellular hosts and comprises in stage (g) the comparison of the expression of the marker gene by each of the two separate cellular hosts.

The invention also relates to an analytical method for determining the tropism of an HIV virus for a cellular receptacle, consisting in carrying out the amplification of stage (b) with the pair of primers SEQ ID No: 19 and SEQ ID No: 20, followed by a second amplification with the pair of primers SEQ ID No: 21 and SEQ ID No: 22, to infect in stage (f) two separate cellular hosts with the viral particles that are obtained in stage (e) and that comprise in stage (g) the comparison of the expression of the marker gene by each of the two separate cellular hosts.

The invention also relates to an analytical method for determining the susceptibility of an HIV virus to an inhibitor compound that targets the co-receptors of HIV-1, consisting in carrying out the amplification of stage (b) with the pair of primers SEQ ID No: 19 and SEQ ID No: 20, followed by a second amplification with the pair of primers SEQ ID No: 21 and SEQ ID No: 22, said inhibitor compound that targets the co-receptors of HIV-1, optionally at different concentrations, to be added or not added during the cultivation of stage (d), to carry out the infection of stage (O) with the viral particles that are obtained in stage (e) on two separate cellular hosts and to compare in stage (f) the expression of the marker gene by each of the two separate cellular hosts.

The invention also relates to an analytical method for determining the infectivity or the replicative capacity of an HIV virus consisting in comparing to stage (g) the expression of the marker gene by the second cellular host that is infected with the viral particles that are obtained by applying stages (a) to (f) to a biological sample of a patient, and the expression of the marker gene by the same second cellular host infected with reference viral particles that are obtained by applying stages (a) to (f) to a sample that contains a reference virus.

Advantageously, the reference viral particles that are obtained from a reference virus are viral particles that are obtained by the application of stages (a) to (f) to a biological sample of the same patient at a stage prior to the therapeutic treatment or before the latter.

The invention also relates to an analytical method for determining the virulence of an HIV virus that consists in carrying out the amplification of stage (b) either with the pair of primers SEQ ID No: 13 and SEQ ID No: 14, followed by a second amplification with the pair of primers SEQ ID No: 15 and SEQ ID No: 16, or with the pair of primers SEQ ID No: 17 and SEQ ID No: 18, followed by a second amplification with the pair of primers SEQ ID No: 18 and SEQ ID No: 16, and to measure, during the infection of stage (f), the cytopathogenic effect that is produced on the second cellular host.

Advantageously, the cytopathogenic effect produced during the infection of stage (f) on the second cellular host is measured by means of cytotoxicity techniques such as the measurement of the induction of syncytia, the induction of apoptosis or by flow cytometry.

The invention also relates to an analytical method for determining the susceptibility of an HIV virus to hydroxyurea, consisting in adding or not adding hydroxyurea, optionally at different concentrations, either during cultivation stage (e), or to the second cellular host and in carrying out in stage (g) the comparison of the expression of the marker gene with and without hydroxyurea.

According to the method of the invention, the duration of cultivation stage (e) is preferably between 12 and 72 hours; quite preferably it is between 24 and 48 hours.

The invention also has as its object a kit for the implementation of an analytical method of a phenotypic characteristic of the HIV viruses that are present in a biological sample of a patient, characterized in that it comprises:
i. a pair of primers that frame a nucleic acid sequence of the viral genome that can carry at least one mutation,
ii. a vector that comprises the parts of a genome of an HIV virus that are necessary to the viral replication except for the segment amplified with the primers that are defined in (i) and the gene that codes for the envelope protein,
iii. a second vector that comprises a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents necessary for carrying out the amplification by PCR,
vii. the products and reagents that make it possible to detect the expressed marker.

Advantageously, the kit according to the invention comprises:
i. the sequence primer pairs:
   SEQ ID No: 1 and SEQ ID No: 2
   SEQ ID No: 3 and SEQ ID No: 4
ii. a retroviral vector that is deleted from the region of the pol reading frame that codes for the HIV-1 protease that extends from residues 1505 to 2565 inclusive, deleted from the envelope region and comprising a unique restriction site M1u1,
iii. a pseudotype virus with a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

Advantageously, the kit according to the invention comprises:
i. the sequence primer pairs:
   SEQ ID No: 5 and SEQ ID No: 7
   SEQ ID No: 6 and SEQ ID No: 8
ii. a retroviral vector that is deleted from the region of the pot reading frame that codes for the HIV-1 reverse transcriptase that extends from residues 2618 to 2872 inclusive and comprises a unique restriction site M1uI,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

Advantageously, the kit according to the invention comprises:
i. the sequence primer pairs:
   SEQ ID No: 9 and SEQ ID No: 10
   SEQ ID No: 11 and SEQ ID No: 12
ii. a retroviral vector that is deleted from the entire region of the pol reading frame that codes for the HIV-1 integrase that extends from residues 4228 to 5093 inclusive and the region that codes for the viral envelope between positions 6343 and 7611 inclusive,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

Advantageously, the kit according to the invention comprises:
1. the sequence primer pairs:
   SEQ ID No: 13 and SEQ ID No: 14
   SEQ ID No: 15 and SEQ ID No: 16
ii. a retroviral vector that is deleted from the entire region that codes for the extracellular portion of sub-unit gp41 of the HIV-1 envelope that extends from residues 7745 to 8263 inclusive and comprises a unique restriction site Mu11,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

Advantageously, the kit according to the invention comprises:
i. the sequence primer pairs:
   SEQ ID No: 17 and SEQ ID No: 14
   SEQ ID No: 18 and SEQ ID No: 16
iii. a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mull,
iv. a first cellular host that can be infected by an HIV virus,
v. a second cellular host that can be infected by an HIV virus and that comprises a marker gene that can be activated only by viral particles,
vi. the products and reagents that are necessary for carrying out the amplification by PCR,
vii. the products and reagents that make it possible to detect the expressed marker.

Advantageously, the kit according to the invention comprises:
1. the sequence primer pairs:
   SEQ ID No: 19 and SEQ ID No: 20
   SEQ ID No: 21 and SEQ ID No: 22
ii. a retroviral vector that is deleted from the region, coding for the domains that extend from loop VI to loop V3 of the HIV-1 envelope that extends from 6617 to 7250 inclusive and comprises a unique restriction site NheI,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

Advantageously, the kit according to the invention comprises:
i. the sequence primer pairs:
   SEQ ID No: 23 and SEQ ID No: 24
   SEQ ID No: 25 and SEQ ID No: 26 with SEQ ID No: 27
ii. a retroviral vector that is deleted from the entire region that codes for the extracellular portion of sub-unit gp41 of the HIV-1 envelope that extends from residues 7745 to 8263 inclusive and comprises a unique restriction site Mu11,
iv. a first cellular host that can be infected by an HIV virus,
v. a second cellular host that can be infected by an HIV virus and that comprises a marker gene that can be activated only following viral infection,
vi. viithe products and reagents that are necessary for carrying out the amplification by PCR,
vii. the products and reagents that make it possible to detect the expressed marker.

Advantageously, the kit according to the invention comprises:
1. the sequence primer pairs:
   SEQ ID No: 17 and SEQ ID No: 24
   SEQ ID No: 18 and SEQ ID No: 26 and SEQ ID No: 27
ii. a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mull,
iv. a first cellular host that can be infected by an HIV virus,
v. a second cellular host that can be infected by an HIV virus and that comprises a marker gene that can be activated only by viral particles,
vi. the products and reagents that are necessary for carrying out the amplification by PCR,
vii. the products and reagents that make it possible to detect the expressed marker.

### BRIEF DESCRIPTION OF THE FIGURES

Other advantages and characteristics of the invention are illustrated in the following examples that refer to the following figures:

FIG. 1 diagrammatically shows the plasmid pSRT. The region that codes for the reverse transcriptase of pNL4-3xcenv is deleted by means of digestion by Ba₁I-SnaB1. The linearization of the resulting pSRT is accomplished by using Nru I.

FIG. 2 illustrates the curves of the dose response effects obtained for two patients versus AZT and 3TC, before and after treatment with inhibitors of reverse transcriptase.

The details of the treatment, the sampling relative to the time, and the genotypes are found in the headings of the figures.

The curves show the inhibition of the infection by a recombinant virus of P4 cells treated either with ziodvudine (AZT, sample groups A and C) or lamivudine (3TC, sample groups B and D), according to the technique that is described later, in the Equipment and Methods paragraph. For patient I, the samples were tested at 0 (♦), 9 (□) and 18 (□) months after treatment, and for patient 2 at 0 (♦) and 27 (●) months after treatment.

FIG. 3 is a diagram of the first stages (a and b) of a particular implementation of the method of the invention. The diagram illustrates the stages of extraction (a) and amplification (b) of the reverse transcriptase sequences that are extracted from the plasma of a patient by RT PCR of the method of the invention as well as the diagrams of plasmid pRVA/RT constructions used later in stage (d).

FIG. 4 is a diagram of stages (c) to (g) of a particular implementation of the method of the invention. This diagram illustrates stage (d) of co-transfection in HeLa/293T cells of the amplified nucleic acids of stage (b), of a first plasmid p43xcsnΔenv RT constructed from a genome of an HIV virus, not comprising a nucleic acid segment that corresponds to all or part of the amplified nucleic acid sequence in stage (b) nor a fragment of the nucleic acid sequence that codes for the envelope protein and a second pVSV-G plasmid that comprises the sequence that codes for the envelope protein; whereby cultivation stage (e) makes it possible to produce viral particles, stage (f) for transfection of viral particles that are obtained in stage (e) into P4 cells, previously incubated with or without the presence of serial dilutions of different inhibitors of reverse transcriptase, whereby said P4 indicating cells comprise a system for expression of the gene that codes for the beta-galactosidase enzyme that can be activated only by the activation sequences tat that are expressed by the recombinant virus, and stage (g) for detection and/or quantification of the beta-galactosidase by means of substrate CPRG.

FIG. 5 illustrates the results of the phenotypic analysis obtained in the presence of the T20 fusion inhibitor for viruses that are present in the plasmas of patients (P1, P2 and P3) and reference viral prototypes (N L 43 and the resistant clone DIM).

FIG. 6 illustrates the results of the measurement of the replicative capacity of viruses L1, L2 and L3 that are extracted from different samples.

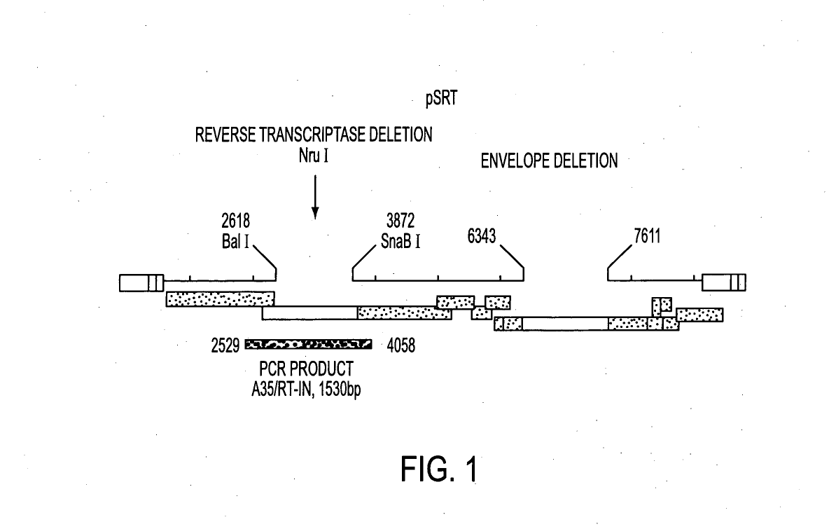

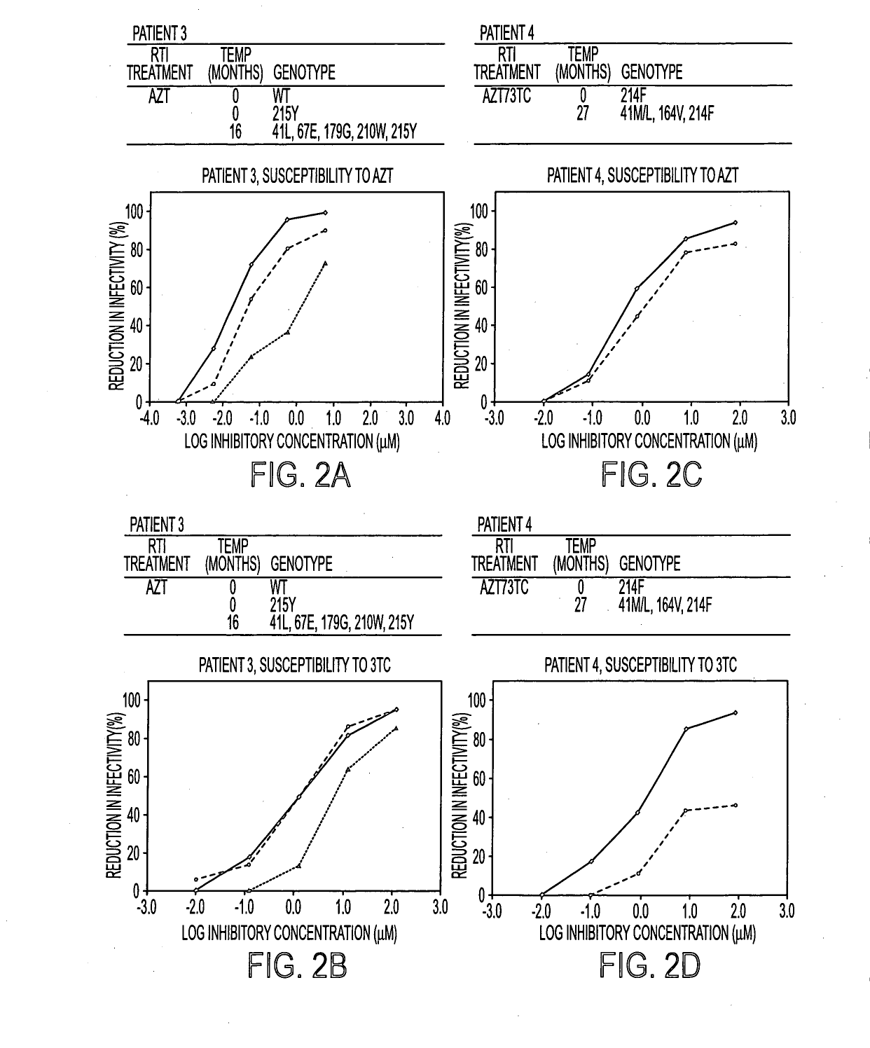

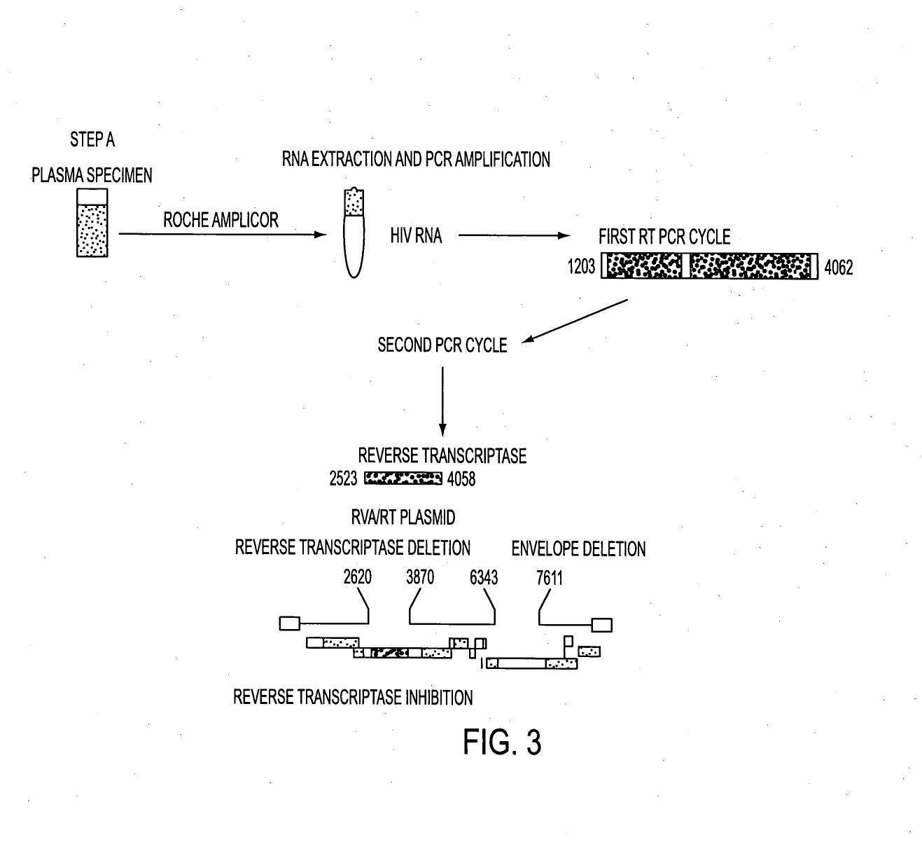

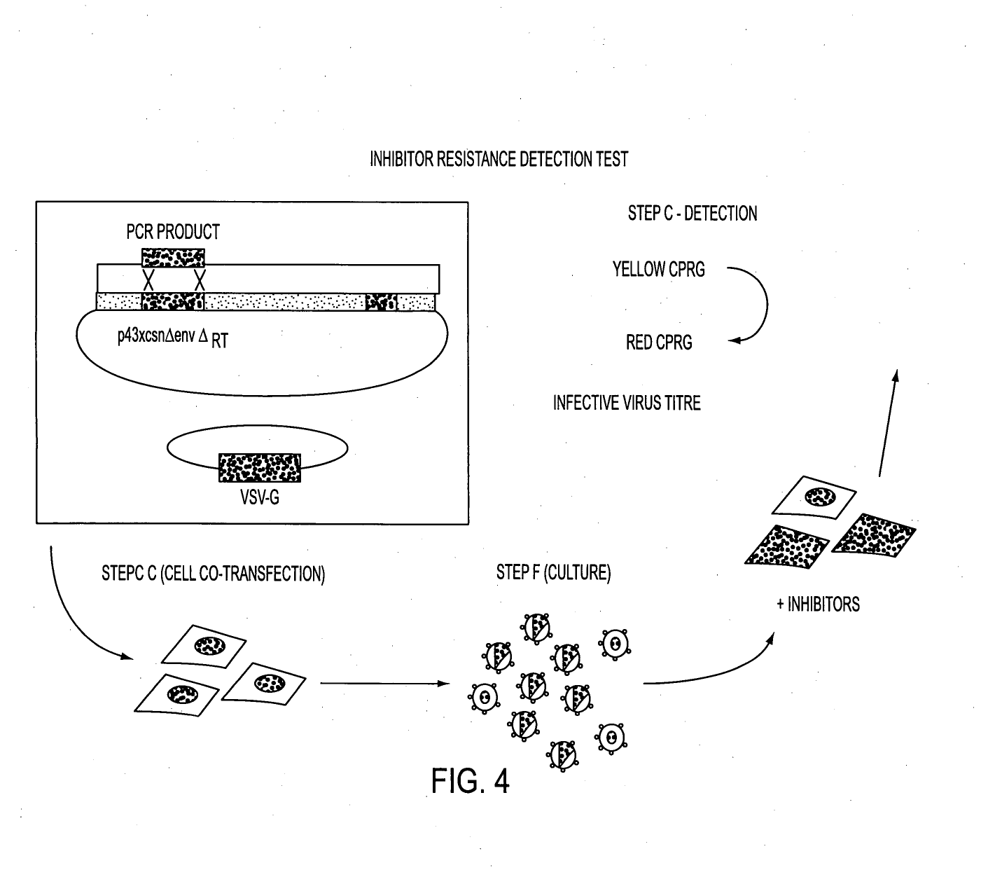

### EXAMPLES

I. Equipment and Methods

1.1—Amplification by Chain Polymerization Reaction (PCR).

The RNA is isolated from the plasma of the patients by means of a Roche Amplicor® tool kit (Roche Diagnostics, 38242 Meylan Cedex, France), and the genes of interest ate isolated by means of a reverse transcriptase and a subsequent PCR reaction.

The amplification of the region that codes for the reverse transcriptase (RT) is carried out by means of external primers MJ3 (5′ AGT AGG ACC TAC ACC TGT CA 3′) (sequence SEQ ID NO: 5, attached) and RT-EXT (5′ TTC CCA ATG CAT ATT GTG AG 3′) (sequence SEQ ID NO: 6, attached) and internal primers A35 (5′ TTG GTT GCA TAA ATT TTC CCA TTA GTC CTA TT 3′) (sequence SEQ ID No: 7, attached) and RT-IN (5′ TTC CCA ATG CAT ATT GTG AG 3′) (sequence SEQ ID No: 8, attached) with an initial cycle at 50° C. (30 minutes) and at 94° C. (2 minutes), followed by 40 cycles at 94° C. (30 seconds), 55° C. (30 seconds) and 68° C. (90 seconds), and a final extension stage at 98° C. for 10 minutes. This amplifies a product of 1530 pb that extends beyond codon 93 of the region that codes the protease and beyond codon 503 of the region that codes the polymerase (pol).

The products that are thus obtained by PCR are purified on QuiaAmp® columns and analyzed regarding their size, their degree of purity and their approximate concentration by electrophoresis on agarose gels.

1.2—Genotyping

The sequences of nucleotides of the coding regions of the reverse transcriptase are determined by automatic sequencing of the termination of the dideoxynucleotide chain of the crude PCR products.

1.3—Plasmids

The molecular clones of the HIV-1 used in the analytical method are derived from pNL4-3.

The plasmid that is deleted by reverse transcriptase is constructed by a modification of the pNL4-^{3xcΔenv mut},e to bring unique SnaBI restriction sites into position 3872 and Nru I into position 3892.

Enzymes Ba I and SnaB I are used to withdraw the coding region for reverse transcriptase (between positions 2618 and 2872), and the linearization of the resultant plasmid pSRT is carried out by means of the enzyme Nru L The expression of the envelope glycoprotein VSV-G in the transfected cells is ensured by the pVSV plasmid that contains the coding sequence vsv-g under the monitoring of a CMV promoter.

1.4—Cellular Cultures.

HeLa, 293 T and P4 cells are cultivated in the DMEM medium that is supplemented with 10% fetal calf serum (FCS), 50 U/ml of penicillin and 50 μg/ml of streptomycin. The P4 cells are Hela-CD4 and LTR-LacZ cells in which the expression of the beta-galactosidase can be induced only by the transactivating Tat protein of HIV, consequently making possible a precise quantification of infectivity or of the replicative capacity of the HIV-1 viruses based on a unique replication cycle (Charneau, P.; Mirambeau, G.; Roux, P.; Paulous, S.; Buc, H. and Clavel, F. (1994) “HIV-1 Reverse Transcription. A Termination Step at the Center of the Genome.” J Mol Biol 241 (5), 651-62). The P4 cells are cultivated in the presence of 500 μg/ml of geneticin.

II.—Determination of the Susceptibility of an HIV Virus to a Reverse Transcription (RT) Inhibitor.

The determination of the susceptibility of an HIV virus to a reverse transcriptase inhibitor is carried out in the following manner: 293 T cells are transfected with 7.5 μg of pSRT plasmid that is linearized by NruI, 0.1 μg of pVSV-G plasmid and 0.5 and 1 μg of the product that is obtained from the PCR reaction of the reverse transcription of HIV. The transfection precipitate is withdrawn from cells after 18 hours of incubation, and fresh growth medium is added. After 24 hours of cultivation, the supernatant is clarified by centrifuging (500 g, 15 minutes) and transferred to P4 indicating cells that were pre-incubated with serial dilutions of a reverse transcriptase inhibitor, in triplicate wells, for four hours. The range of inhibitor concentrations used varies according to the compounds. The signal that is produced by the activation of the marker gene was developed with CPRG for 48 hours as for the analysis of the susceptibility to a reverse transcriptase inhibitor, and index IC₅₀ was calculated by using the median effect equation.

III—Optimization of the Analytical Method to Determine the Susceptibility of an HIV Virus to Reverse Transcriptase Inhibitors,

The pSRT plasmid that brings a deletion in the region that codes for pol extending from codon 24 of reverse transcriptase (base 2618) to codon 432 of reverse transcriptase (base 3872) includes all of the mutations combined with a resistance phenomenon that are known to date. The homologous sequences of the reverse transcriptase product obtained from PCR extend 88 base pairs upstream and 186 bases downstream from the deletion in pSRT. The transfections for determining the susceptibility to inhibitors of reverse transcriptase are carried out with a cellular line 293T that has a strong capacity to be transfected rather than with HeLa cells. This does not constitute a problem because the cells are eliminated from the supernatant that contains the virus by centrifuging before the transfer of P4 cells.

The transfer conditions are optimized by means of a checkerboard test. The variation of the ratio of plasmid/product obtained from PCR does not significantly modify the amount of p24 or reverse transcriptase produced or the reaction speed with CPRG. Since the circular plasmid, pVSV-G, seems to be extremely toxic for 293T cells, the amount of the latter was reduced from 3 μg to 0.1 μg in the transfection mixture, leading to high yields of p24 (>20 ng/ml compared to 9.8 ng/ml).

Since the early phases of the replication of the virus, including the reverse transcription, are produced in P4 indicating cells in this type of determination, these are these cells that are treated with serial dilutions of the reverse transcriptase inhibitors.

The inhibitor concentration ranges that are used are selected based on the cellular toxicity of each compound and the IC₅₀/IC₉₀, ratio for the susceptibility of the resistant isolates (Table 1). For example, since index IC₅₀ for abacavir for the P4 cells is about 250 μM whereas the IC₅₀ index for this compound for the native stock of the virus is about 3 μm, the detection of the resistance is limited by the toxicity. A range of four serial dilutions, beginning at 200 μm, was used for the abacavir, making it possible to detect up to 60× more resistance.

In contrast, since the toxicity of the AZT for the P4 cells is high (>300 μm), whereas the IC₅₀ index is considerably lower, a wider range of dilutions was used (dilution in a 1/10 series from 5 μm) so as to allow the detection of high resistance levels (up to 100×).

For the reverse transcriptase RVA, the IC₅₀ index is used rather than the IC₉₀ index because the detection of the IC₉₀ index for the resistant viruses could require toxic compound levels for most of the reverse transcriptase inhibitors.

TABLE 1
Susceptibility of Reference Virus NL4-3 to Reverse Transcriptase
Inhibitors (RTI).

| | Concentrations of | | | | |
|---|---|---|---|---|---|
| | Pharmaceutical | | Geometric | | Maximum |
| RT | Agents Used Ranges/ | | Mean | Standard | Detectable |
| Inhibitors | Stages of Dilution | | IC₅₀^{a} | Deviation^{a} | Susceptibility^{b} |
| AZT | 50 μm-5 nm | 10× | 0.018 μm | 2.7 | 2700 |
| 3TC | 200-0.02 μm | 10× | 1.512 μm | 2.2 | 130 |
| D4T | 100-0.01 μm | 10× | 0.444 μm | 2.7 | 220 |
| DDI | 100-0.01 μm | 10× | 1.613 μm | 2.5 | 60 |
| Abacavir | 200-0.8 μm | 4× | 2.229 μm | 1.8 | 90 |
| Efavirenz | 100-0.16 μm | 5× | 0.716 nm | 2.2 | 140 |
| Nevirapine | 50 μm-5 nm | 10× | 0.037 μm | 2.0 | 1300 |

A—Geometric mean and standard deviation of 20 tests repeated for the reverse transcriptase inhibitors.

B—Approximate maximum difference for the IC₅₀ and IC₉₀ indices between NL43 and the tested virus that can be measured by using the given range of pharmaceutical agent concentrations.

The analytical method for determining the susceptibility of the HIV virus to reverse transcriptase inhibitors provides a standard deviation of the geometric mean for 20 tests between 1.78 (abacavir) and 2.7 (D4T and AZT). The median standard deviation for the reverse transcriptase inhibitors (RTI) tested (AZT, 3TC, D4T, DDI, abacavir, efavirenz and nevirapine) is 2.2. To simplify the automatic interpretation of the results from samples of the patients, an arbitrary Resistance Index (RI) value, RI=5, was defined as the minimum reduction of the susceptibility to an RTI under consideration as being significantly reduced relative to NL43.

To determine if NL43 is a reference virus that is suitable for comparison with clinical isolates, a group of samples taken from patients with ordinary treatment was tested on 3 RVA replicates for their susceptibility to reverse transcriptase inhibitors.

The median IC₅₀ found for 22 tested viruses tends to be slightly higher than the one found for the NL43 virus with an RI media around 0.92 (for stavudine) and 1.22 (lamivudine). Although the RI is less than the defined limit of 5 on the inhibitor total for most of the samples tested, the RI range seems broad, in particular for the non-nucleoside inhibitors. In particular, a resistance index of 11.0 for the nevirapine was obtained for a virus, however, this virus carried a mutation on codon 98 (A for S) of reverse transcriptase, which was previously involved in the resistance to NNRTIs.

IV—Reproducibility

The reproducibility of the method for determining the susceptibility of the recombinant virus (RVA) to reverse transcriptase inhibitors (RTI) was evaluated by a series of test on 5 samples, selected so as to represent a wide range of susceptibility profiles, between 4 and 8 tests per sample, by using RNA preparations and separate PCR reactions.

The inter-test variation for determining the susceptibility of the HIV viruses to reverse transcriptase inhibitors indicates that in some cases, there is a difference that is greater than 5 between the maximum RI and the minimum RI found for repeated determinations (Table 2), however, the standard deviation of the geometric mean is kept 2.2 in all of the cases except one (R4, AZT). In three of the samples, the RI obtained during repeated tests varies between <5 and >5 for the compounds for which the viruses can be moderately resistant (RI not higher than 12).

TABLE 2
Reproducibility of the Susceptibility to RTI.

Sample

| (Number | | Resistance Index^{b} | | | | | | | |
|---|---|---|---|---|---|---|---|---|---|
| of Tests) | Genotype^{a} | | AZT | 3TC | D4T | DDI | Abacavir | Efavirenz | Nevirapine |
| R1 | 69N/T, | MG^{c} | 1.5 | 1.3 | 1.4 | 1.4 | 1.1 | 1.0 | 2.2 |
| (6) | 70R | SD^{d} | 1.5 | 1.5 | 1.4 | 1.6 | 1.2 | 1.1 | 1.8 |
| | | max/min^{e} | 2.9 | 2.8 | 2.2 | 2.8 | 1.6 | 1.2 | 3.8 |
| | | n > 4/N^{f} | 0/6 | 0/6 | 0/6 | 0/6 | 0/6 | 0/6 | 0/6 |
| R2 | 41L, 62V, | MG^{c} | 1232.6 | >f | 43.5 | 26.9 | > | 74.9 | > |
| (6) | 67N, | SD^{d} | 1.6 | na | 2.1 | 1.7 | na | 1.7 | na |
| | 69N, 75I, | max/min^{e} | 3.8 | na | 8.1 | 4.1 | na | 3.9 | na |
| | 77L, | n > 4/N^{f} | 6/6 | 6/6 | 6/6 | 6/6 | 6/6 | 6/6 | 6/6 |
| | 115F, | | | | | | | | |
| | 116Y, | | | | | | | | |
| | 151M, | | | | | | | | |
| | 181C, | | | | | | | | |
| | 184V, | | | | | | | | |
| | 190A, | | | | | | | | |
| | 208Y, | | | | | | | | |
| | 215F, | | | | | | | | |
| | 219Q | | | | | | | | |
| R3 | 184V, | MG^{c} | 13.2 | > | 3.2 | 3.1 | 4.3 | 1.0 | 1.2 |
| (6) | 215F | SD^{d} | 2.7 | na | 1.8 | 1.6 | 1.4 | 1.0 | 1.3 |
| | | max/min^{e} | 8.9 | na | 5.7 | 3.3 | 2.4 | 1.0 | 1.6 |
| | | n > 4/N^{f} | 5/6 | 1/6 | 1/6 | 1/6 | 4/6 | 0/6 | 0/6 |
| R4 | 41L, 67N, | MG^{c} | 137.5 | > | 3.6 | 3.0 | 6.1 | 149.8 | > |
| (5) | 69D, | SD^{d} | 1.8 | na | 2.0 | 1.3 | 1.6 | 1.6 | na |
| | 184V, | max/min^{e} | 4,5 | na | 4,1 | 1,7 | 3,3 | 3,9 | na |
| | 190A, | n > 4/N^{f} | 5/5 | 5/5 | 3/5 | 5/5 | 3/5 | 5/5 | 5/5 |
| | H208Y, | | | | | | | | |
| | 210W, | | | | | | | | |
| | 215Y | | | | | | | | |
| R5 | 215Y, | MG^{c} | 3.3 | 1.8 | 1.7 | 2.6 | 1.7 | > | 580.5 |
| (5) | 41L, 74V, | SD^{d} | 1.6 | 1.7 | 1.8 | 1.6 | 1.6 | na | 1.8 |
| | 100I, | max/min^{e} | 3.0 | 3.7 | 3.7 | 2.5 | 2.9 | na | 2.2 |
| | 103N | n > 4/N^{f} | 1/5 | 0/5 | 0/5 | 0/5 | 0/5 | 5/5 | 5/5 |

^{a}Only the amino acid substitutions already known to be combined with a resistance to reverse transcriptase inhibitors are indicated.
^{b}The Resistance Index is the ratio of IC₅₀ in the sample relative to that of NL43 that is determined in parallel.
^{c}Geometric mean.
^{d}Standard deviation of the geometric mean.
^{e}The highest resistance index obtained in the tests divided by the lowest.
^{f}The number of tests providing a resistance index >4 classifying the virus as resistant/in the total number of tests carried out.
^{g}An IC₅₀ above the range that can be detected in all of the tests is indicated by >. In these cases, a standard deviation and a minimum/maximum are not applicable (na).

The phenotypic results that are obtained for these reverse transcriptase inhibitors show an agreement with the genotypic profiles of the samples.

Sample R2, which shows a high degree of resistance relative to all of the compounds tested, comprises multiple mutations by including those of the multi-compound resistance complex (62V, 75I, 77L, 116Y and 151M) that imparts the resistance to RTI nucleotides, the 3TC resistance combined with the 184V mutation and mutations that are known to induce a reduced susceptibility to NNRTI (181 C, 190A).

Sample R3 shows a high resistance level at 3TC, again modulated by the mutation 184V, with a considerable variation of the RI for AZT that probably reflects the inconsistent nature of the suppression of the resistance that is induced by 215F by 184V.

In sample R4, such a suppression is thwarted by the presence of multiple mutations including a mutation 208Y.

Sample R5 is kept approximately at AZT despite a mutation 41L and a mutation 215Y because of the effects of the mutation 1001, which, in combination with 103N, is responsible for the high resistance levels observed with regard to efavirenz and nevirapine.

V—Validation of the Analytical Method for Determining the Susceptibility of an HIV Virus to Fusion Inhibitors.

The recombinations that are obtained by using for the amplification of stage (b) the pair of primers FuA: 5′ AAGCAATGTATGCCCCTCCCAT3′ (SEQ ID No: 23) and FuB: 5′ GGTGGTAGCTGAAGAGGCACAGG3′ (SEQ ID No: 24) followed by a second amplification, carried out with the primer: FuC: 5′ ATATGAGGGACAATTGGAGAAGTGA3′ (SEQ ID No: 25) and a mixture of the following primers: FuD1: 5′ TCTGTCTCTCTCTCCACCTTCTTCTT3′ (SEQ ID No: 26) and FuD2: 5′ TCTGTCTTGCTCTCCACCTTCTTCTT3′ (SEQ ID No: 27) are very effective.

Actually, to obtain a satisfactory viral production, 10-50 ng of PCR product, used in the recombination stage, is generally adequate. In this method that is intended to analyze the phenotypic fusion characteristic, a viral production corresponding to 50-400 ng/ml of p24 is obtained after transfection. For the infection of target cells, between 0.5 and 5 ng of p24/well (96-well plates) is used. The increase of the optical density that is obtained after infection of target cells is linear if the infection is conducted under these conditions.

To validate the analytical method that relates to the susceptibility of HIV viruses to fusion inhibitors, two fusion inhibitors have been used: a peptide derived from the distal helix sequence in the gp41 of HIV (called DP178 or T20) and a derivative of betulinic acid (RPR103611). For each inhibitor, the reduction of sensitivity of one or more resistant viruses (previously identified by other authors) relative to two reference viruses: a primary plasmatic virus T5A1 and a virus adapted to in-vitro cultivation (LAI) was carried out. All the viruses were produced by recombination.

Results

V.1—DP178 or T20 Inhibitor

The increases of IC50 for a strongly resistant NL-DIM virus (described by Rimsky, L. T. et al. J. Virol. 1998, Vol. 72, pages 896-993) and for the NL4.3 virus (which is partially resistant) were measured regarding this inhibitor.

The strongly resistant DIM virus exhibits an increase of the value of IC50 by 80× relative to T5A1 and more than 100×relative to LAI.

The partially resistant NL4.3 virus is characterized by an increase of IC50 by 10× relative to 15A1 and 12.5× relative to LAI.

V.1.1 Determination of the Susceptibility of a Plasmatic HIV to the T20 Fusion Inhibitor

The viral RNA is extracted from plasmas of HIV-positive patients.

A first amplification is carried out by PCR with the pair of primers ED3_E01-.

A second amplification is carried out by PCR with the pair of primers E10_FuB to obtain a DNA fragment of 2200 base pairs that extend between residues 6322 and 8522.

The transfection of a first cellular host is carried out with:
the nucleic acids that are obtained after the second amplification.
the retroviral vector that is deleted from the entire region that codes for the majority of the gp120 sub-unit and the extracellular portion of the gp41 of the HIV envelope that extends from residues 6480 to 8263 inclusive and comprises a unique restriction site MluI.

The first cellular host is cultivated under conditions that make it possible to produce viral particles.

The infection is carried out by viral particles that are produced in the preceding stage from a second cellular host (comprising a marker gene that can be activated following viral infection) in the presence of an increasing dose of T20 fusion inhibitor.

The quantification of the marker that is expressed is carried out so as to demonstrate the level of susceptibility of the HIV viruses that are present in the biological samples to the T20 fusion inhibitor.

The results of the phenotypic analysis described above for viruses that are present in plasmas of patients (P1, P2 and P3) and reference viral prototypes (NL43 and resistant clone DIM) are illustrated in FIG. 5, attached, and the IC50 indices are shown in Table 3 below.

TABLE 3

| Sample | IC50 |
|---|---|
| P1 | 0.132 |
| P2 | 0.017 |
| P3 | 0.047 |
| DIM | >3 |
| NL 43 | 0.234 |

V.2—RPR103611 Inhibitor

The increase of IC50 of the LA1-L91H resistant virus (Labrosse, B. et al. J. Virol. 2000 Vol 74, pages 2142-2150) regarding this inhibitor has been measured.

For the LAT-L91H virus, the increase of IC50 relative to LAI and to T5A1 is greater than 100×.

VI—Optimization of the Analytical Method for Determining the Neutralizing Capacity of the HIV Viruses

The recombinations that are obtained by using for the amplification of stage (b) a pair of primers NEU-A: 5′ TAGAAAGAGCAGAAGACAGTGGCAATG3′ (SEQ ID No: 17) and FuB: 5′ GGTGGTAGCTGAAGAGGCACAGG3′ (SEQ ID No: 24), followed by a second amplification, with the primers: NEU-C: 5′ GTGGGTCACAGTCTATTATGGGG3′ (SEQ ID No: 18) and a mixture of primers FuD1: 5′ TCTGTCTCTCTCTCCACCTTCTTCTT3′ (SEQ ID No: 26) and FuD2: 5′ TCTGTCTTGCTCTCCACCTTCTTCTT3′ (SEQ ID No: 27) are also very effective.

Actually, during monitoring of viral production, it has turned out that to obtain a satisfactory viral product, generally 10-50 ng of PCR product, used in the recombination stage, is adequate.

A viral production corresponding to 50-400 ng/ml of p²⁴ after transfection is obtained. For the infection of target cells, between 0.5 and 5 ng of p24/well (96-well plates) is used. The increase in the optical density obtained after infection of target cells is linear if the infection is conducted under these conditions.

VII. Measurement of Viral Infectivity (or Replicative Capacity Test)

A first amplification by PCR is carried out with the pair of primers FITA and PROA−.

A second amplification by PCR is carried out with the pair of primers FITB and PROB− to obtain a DNA fragment of 1488 base pairs that extend between residues 1237 and 2725.

The transfection of a first cellular host is carried out with:
the nucleic acids that are obtained after the second amplification.
the retroviral vector that is deleted from a region that codes for a portion of Gag and the entire protease and that comprises a unique restriction site M1uI.

The cultivation of the first cellular host is carried out under conditions that make it possible to produce viral particles.

The amount of recombinant viral particles is measured in the supernatant of this culture by an Elisa test metering the amount of p²⁴ antigen.

The infection is carried out by the viral particles that are produced in the preceding stage from a second cellular host (comprising a marker gene that can be activated following viral infection) by increasing dilutions of viral supernatant.

The quantification of the marker that is expressed to demonstrate the infectivity level of the recombinant virus is carried out.

The replicative capacity of the recombinant virus is calculated by plotting a straight regression line from the measured values and by adopting the slope of this straight line.

The viral replicative capacity is expressed relative to that of a reference laboratory virus or relative to the recombinant virus that carries viral sequences that are obtained from the same patient before establishing treatment and before development of the resistance. The results that are obtained are illustrated in FIG. 6, attached.

The L1, L2 and L3 viruses correspond to recombinant viruses that carry protease sequences that are obtained at three different moments from the same patient. The L1 virus carries protease sequences that are obtained before treatment. Each point corresponds to the optical density that is measured after infection of P4 indicating cells by a dilution of each of the 3 viruses. A straight line of regression is traced for the series of measurements of each virus. The slope of the straight line is calculated. The replicative capacity of L2 is expressed relative to the slope of L2 to that of L1, and the replicative capacity of L3 by the ratio of the slope of L3 to that of L1. Here, the reference virus is the pretherapeutic virus L1. When the pretherapeutic virus is lacking, it is possible to express the replicative capacity of a patient virus relative to a reference laboratory virus such as pNL4-3.

1. A method for analysis of a phenotypic characteristic of HIV viruses that are present in a biological sample from a patient, whereby said phenotypic characteristic results from one or more mutations of the viral genome that can influence the viral infection, wherein said method comprises:
a) extracting nucleic acids that are contained in said biological sample,
b) amplifying by PCR a segment of the nucleic acids of stage (a), each with a pair of primers that frame a nucleic acid sequence of the viral genome that can carry at least one mutation,
c) preparing a vector that comprises the parts of a genome of an HIV virus that are necessary for the viral replication except for the amplified segment in stage (b),
d) transfecting a first cellular host with:
The nucleic acids that are obtained in stage (b),
The vector that is prepared in stage (c),
to obtain a chimerical virus by homologous recombination,
e) culturing said first cellular host under conditions that make it possible to produce viral particles during a single replication cycle,
f) infecting said first cellular host with viral particles that are obtained in stage (e) from at least a second cellular host that can be infected by an HIV virus or an HIV-pseudotype virus,
g) detecting, quantifying, or both, the marker that is expressed in stage (f) so as to demonstrate at least one characteristic of the HIV viruses that are present in the biological sample.

2. The method of claim 1, wherein the amplifying by PCR of stage (b) is carried out with a pair of primers that frame a nucleic acid sequence that comprises all or part of a viral genome region that is selected from among: gag, pol, protease, reverse transcriptase, RNAse H, integrase, vif, vpr, tat, rev, vpu, env, nef, cis-active sequences, LTR, dimerization sequences, splicing-regulating sequences or the Rev response element (RRE).

3. The method of claim 1, wherein the amplifying by PCR of stage (b) is carried out with a pair of primers that frame a nucleic acid sequence that codes for a portion of the gag protein of the human immunodeficiency virus and a nucleic acid sequence that codes for the protease that can carry at least one mutation in the gene that codes for the protease and wherein the vector of stage (c) is constructed from a genome of an HIV virus where all or part of the gene that codes for the protease is deleted.

4. The method of claim 1, wherein the amplifying of stage (b) that carries at least one mutation in the gene that codes for the protease is made with a pair of primers:
(SEQ ID No: 1)

| | |
|---|---|
| Fit A-: | (5′ TCA CCT AGA ACT TTA AAT GC 3′) and |

(SEQ ID No: 2)

| | |
|---|---|
| Pro A-: | (5′ GGC AAA TAC TGG AGT ATT GTA TG3′ 3′), |

followed by a second amplification with a pair of primers:

| | |
|---|---|
| (SEQ ID No:3) | |
| Fit B: | (5′ AGA ACT TTA AAT GCA TGG GT 3′ ) and |
| (SEQ ID No:4) | |
| Pro B-: | (5′ GGA GTA TTG TAT GGA TTT TCA GG 3′) , |

to obtain a DNA segment of 1488 base pairs that extend between residues 1237 and 2725 inclusive, and
wherein the vector of stage (c) is a retroviral vector that is deleted from the region of the pol reading frame that codes for the HIV-1 protease that extends from residues 1505 to 2565 inclusive, deleted from the envelope region and comprising a unique restriction site M1uI.

5. The method of claim 1, wherein the amplifying by PCR of stage (b) is carried out with a pair of primers that frame a nucleic acid sequence that can carry at least one mutation in the gene that codes for the reverse transcriptase, and the transfecting of stage (c) is carried out with a first vector that is constructed from a genome of an HIV virus where all or part of the gene that codes for the reverse transcriptase is deleted.

6. The method of claim 1, wherein the amplifying of stage (b) is carried out with a pair of primers:
(SEQ ID No: 5)

| | |
|---|---|
| MJ3 | (5′ AGT AGG ACC TAC ACC TGT CA 3′) and |

(SEQ ID No: 6)

| | |
|---|---|
| RT-EXT | (5′ TTC CCA ATG CAT ATT GTG AG 3′), |

followed by a second amplification stage with a pair of primers:
A35 (5′ TTG GTT GCA TAA ATT TTC CCA TTA GTC CTA TT 3′) (SEQ ID No: 7) and
RT-IN (5′ TTC CCA ATG CAT ATT GTG AG 3′) (SEQ ID No: 8)
to obtain a DNA segment of 1530 base pairs that extend beyond codon 93 of the region that codes for the protease and beyond codon 503 of the region that codes for the polymerase (POL), and
wherein the vector of stage (c) is a retroviral vector that is deleted from the region of the pol reading frame that codes for the HIV-1 reverse transcriptase that extends from residues 2618 to 2872 inclusive and comprises a unique restriction site MluI.

7. The method of claim 1, wherein the amplifying by PCR of stage (b) is carried out with a pair of primers that frame a nucleic acid sequence that codes for a portion of the gag protein, for the protease and for a portion of the reverse transcriptase of the human immunodeficiency virus that can carry at least one mutation in the nucleic acid sequence that codes for the gag protein or for the protease or for the reverse transcriptase.

8. The method of claim 1, wherein the amplifying of stage (b) is carried out with the pair of primers:
gag+1 (5′ AGGGGCAAATGGTACATCA 3′) (SEQ ID No: 31) and
RT-EX (SEQ ID No 6),
followed by a second amplification stage with a pair of primers:
Fit B+ (SEQ ID No: 1) and
RT-IN (SEQ ID No: 8)
to obtain a DNA segment of 2825 base pairs that extend between residues 1237 and 4062 and
wherein the transfecting of stage (c) is carried out with a retroviral vector that is deleted from a portion of the gag gene and regions within the pol reading frame that codes for the protease and a portion of the HIV-1 reverse transcriptase that extends from residues 1507 to 3870 inclusive, deleted in the envelope region and comprising a unique restriction site NruI.

9. The method of claim 1, wherein said method further comprises determining the susceptibility of an HIV virus to an inhibitor compound of reverse transcriptase, wherein said inhibitor compound of reverse transcriptase is added or not added to the second cellular host, prior to the infection of the latter by viral particles that are obtained in stage (e), and wherein stage (g) comprises the comparison of the expression of the marker gene with and without an inhibitor compound of the reverse transcriptase.

10. The method of claim 1, wherein the amplifying by PCR of stage (b) is carried out with a pair of primers that frame a nucleic acid sequence that can carry at least one mutation in the gene that codes for integrase, and the vector of stage (c) is a retroviral vector that is deleted from all or part of the gene that codes for integrase.

11. The method of claim 1, wherein the amplifying of stage (b) is carried out with the pair of primers:

| | | |
|---|---|---|
| INT B+ - | 5′GTTACTAATAGAGGAAGACAAA3′ | (SEQ ID No: 9) |
| and | | |
| INT B− | 5′TTTTGGTGTTATTAATGCT3′, | (SEQ ID No: 10) |

followed by a second amplification stage, with the pair of primers:

| | |
|---|---|
| INT V+ | |
| 5′ CACCCTAACTGACACAACAA3′ and | (SEQ ID No:11) |
| INT V− | |
| 5′ AAGGCCTTTCTTATAGCAGA3′, | (SEQ ID No:12) |

to obtain a DNA fragment of 1460 base pairs that extend from residues 3950 to 5410 inclusive,
wherein the vector of stage (c) is a retroviral vector that is deleted from the entire region of the pol reading frame that codes for the HIV-1 integrase that extends from residues 4228 to 5093 inclusive and from the region that codes for the viral envelope between positions 6343 and 7611 inclusive.

12. The method of claim 1, wherein said method further comprises determining the susceptibility of an HIV virus to an inhibitor compound of the integrase, comprising adding or not adding said inhibitor compound of the integrase during stage (e), before stage (f), and wherein stage (g) comprises the comparison of the expression of the marker gene with and without an inhibitor compound of the integrase.

13. The method of claim 1, wherein the amplifying by PCR of stage (b) is carried out with a pair of primers that frame a nucleic acid sequence that can carry at least one mutation in the gene that codes for the envelope protein, and wherein the vector of stage (c) is a retroviral vector that is constructed from a genome of an HIV virus where all or part of the gene that codes for the envelope protein is deleted.

14. The method of claim 1, wherein the vector of stage (c) is a retroviral vector that is deleted from the entire region that codes for the extracellular portion of sub-unit gp41 of the HIV-1 envelope that extends from residues 7745 to 8263 inclusive, from the region of the HIV-1 genome that constitutes the Rev response element (RRE).

15. The method of claim 1, wherein the amplifying of stage (b) is carried out with a pair of primers:

| | |
|---|---|
| FIN-A: | |
| 5′ TCAAATATTACAGGGCTGCT3′ and | (SEQ ID No: 13) |
| FIN-B: | |
| 5′ TAGCTGAAGAGGCACAGG3′ | (SEQ ID No: 14) |

followed by a second amplification stage, carried out with the pair of primers:

| | |
|---|---|
| FIN-C: | |
| 5′ CTATTAACAAGAGATGGTGG3′and | (SEQ ID No: 15) |
| FIN-D: | |
| 5′ TCCACCTTCTTCTTCGATT3′, | (SEQ ID No: 16) |

to obtain a DNA segment of 965 base pairs that extend from residues 7553 to 8517 inclusive and
wherein the vector of stage (c) is a retrovital vector that is deleted from the entire region that codes for the extracellular portion of sub-unit gp41 of the HIV-1 envelope that extends from residues 7745 to 8263 inclusive and comprises a unique restriction site Mu11.

16. The method of claim 1, wherein the amplifying of stage (b) with a pair of primers that frame a nucleic acid sequence that can carry at least one mutation in the gene that codes for the envelope protein is made with a pair of primers:

| | |
|---|---|
| FuA: | |
| 5′ AAGCAATGTATGCCCCTCCCAT3′and | (SEQ ID No: 23) |
| FuB: | |
| 5′ GGTGGTAGCTGAAGAGGCACAGG3′, | (SEQ ID No: 24) |

followed by a second amplification stage, carried out with the primer:

| | |
|---|---|
| FuC′: | |
| 5′ ATATGAGGGACAATTGGAGAAGTGA3′ | (SEQ ID No: 25 |

and a mixture of the following primers:

| | |
|---|---|
| FuD1: | |
| 5′ TCTGTCTCTCTCTCCACCTTCTTCTT3′ | (SEQ ID No: 26) |
| and | |
| FuD2: | |
| 5′ TCTGTCTTGCTCTCCACCTTCTTCTT3′, | (SEQ ID No: 27) |

to obtain a DNA segment of 805 base pairs that extend from residues 7635 to 8440 inclusive, and the vector of stage (c) is a retroviral vector that is deleted from the entire region that codes for the extracellular portion of sub-unit gp41 of the HIV-1 envelope that extends from residues 7745 to 8263 inclusive and comprises a unique restriction site Mull.

17. The method of claim 1, wherein the amplifying of stage (b) is carried out with a pair of primers:

| | |
|---|---|
| NEU-A: | |
| 5′ TAGAAAGAGCAGAAGACAGTGGCAATG3′ | (SEQ ID No: 17) |

and

| | | |
|---|---|---|
| FIN-B: | 5′ TAGCTGAAGAGGCACAGG3′, | (SEQ ID No: 14) |

followed by a second amplification stage, with the pair of primers:

| | |
|---|---|
| NEU-C: | |
| 5′ GTGGGTCACAGTCTATTATGGGG3′ | (SEQ ID No: 18) |
| and | |
| FIN-D: | |
| 5′ TCCACCTTCTTCTTCGATT3′, | (SEQ ID No: 16) |

to obtain a DNA fragment of between 2106 and 2320 base pairs that extend from residues 6197-6222 up to residues 6197-6222 inclusive and
wherein the vector of stage (c) is a retroviral vector that is deleted from the entire region that codes for the majority of sub-unit gp120 and the extracellular portion of gp41 of the HIV-1 envelope that extends from residues 6480 to 8263 inclusive and comprises a unique restriction site M1u1.

18. The method of claim 1, wherein the amplifying of stage (b) with a pair of primers that frame a nucleic acid sequence that can carry at least one mutation in the gene that codes for the envelope protein is made with a pair of primers:

| | |
|---|---|
| NEU-A: | |
| 5′ TAGAAAGAGCAGAAGACAGTGGCAATG3′ | (SEQ ID No: 17) |

and

| | | |
|---|---|---|
| FuB: | 5′ GGTGGTAGCTGAAGAGGCACAGG3′, | (SEQ ID No: 24) |

followed by a second amplification stage, with the primers:

| | |
|---|---|
| NEU-C: | |
| 5′ GTGGGTCACAGTCTATTATGGGG3′ | (SEQ ID No: 18) |

and a mixture of the primers

| | |
|---|---|
| FuD1: | |
| 5′ TCTGTCTCTCTCTCCACCTTCTTCTT3′ | (SEQ ID No: 26) |
| and | |
| FuD2: | |
| 5′ TCTGTCTTGCTCTCCACCTTCCTTCTT3′, | (SEQ ID No: 27) |

to obtain a DNA fragment of 2118 base pairs that extend from residues 6322 to 8440 inclusive, and the vector of stage (c) is a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mu11.

19. The method of claim 1, wherein the amplifying of stage (b) with a pair of primers that frame a nucleic acid sequence that can carry at least one mutation in the gene that codes for the envelope protein is made with the pair of primers:

| | |
|---|---|
| ED3: | |
| 5′TTAGGCATCTCCTATGGCAGGAAGAAGCGG3′ | (SEQ ID No: 28) |

and
(SEQ ID No: 29)

| | |
|---|---|
| E01: | 5′ TCCAGTCCCCCCTTTTCTTTTAAAAA3′, |

followed by a second amplification stage, with the primers:

| | | |
|---|---|---|
| E10: | 5′GTGGGTCACAGTCTATTATGGGGT3′ | (SEQ ID No: 30) |
| and | | |
| FuB: | 5′GGTGGTAGCTGAAGAGGCACAGG3′ | (SEQ ID No: 24) |

to obtain a DNA fragment of 2200 base pairs that extend from residues 6322 to 8522 inclusive, and the vector of stage (c) is a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mull.

20. The method of claim 1, wherein the amplifying of stage (b) is carried out with a pair of primers:
(SEQ ID No: 19)

| | |
|---|---|
| E00: | 5′ TAGAAAGAGCAGAAGACAGTGGCAATGA3′ and |

(SEQ ID No: 20)

| | |
|---|---|
| ES8B: | 5′ CACTTCTCCAATTGTCCCTCA3′, |

followed by a second amplification stage with the pair of primers:
(SEQ ID No: 21)

| | |
|---|---|
| E20: | 5′ GGGCCACACATGCCTGTGTACCCACAG3′ and |

(SEQ ID No: 22)

| | |
|---|---|
| E115: | 5′ AGAAAAATTCCCCTCCACAATTAA3′, |

to obtain a DNA segment of 938 base pairs that extend from residues 6426 to 7364 inclusive, and
wherein the vector of stage (c) is a retroviral vector that is deleted from the region, coding for the domains that extend from loop VI to loop V3 of the HIV-1 envelope that extends from 6617 to 7250 inclusive and comprises a unique restriction site NheI.

21. The method of claim 1, wherein said method further comprises determining the susceptibility of an HIV virus to a fusion-inhibitor compound that targets the gp41 protein of HIV-1, wherein said fusion-inhibitor compound is added during the cultivation of the cellular host that is obtained in stage (e), before stage (f) and wherein stage (g) comprises the comparison of the expression of the marker gene with and without a fusion inhibitor compound targeting the gp41 of HIV-1.

22. The method of claim 1, wherein said method further comprises determining the susceptibility of an HIV virus to a compound that inhibits the entry of said HIV virus into a target cell, wherein said compound that inhibits entry is added to the cellular host that is obtained in stage (e) before the infection of stage (f) and wherein stage (g) comprises the comparison of the expression of the marker gene with and without a compound that inhibits entry.

23. The method of claim 1, wherein said method further comprises determining the susceptibility of an HIV virus to the inhibiting action of antibodies, wherein said method is carried out without antibodies and with antibodies whereby said antibodies are present in stage (e) and wherein stage (g) comprises the comparison of the expression of the marker gene with and without antibodies.

24. The method of claim 1, wherein said method further comprises determining the tropism of an HIV virus for a cellular receptor, wherein the infection of stage (f) with the viral particles that are obtained in stage (e) is carried out on two separate cellular hosts, and stage (g) comprises the comparison of the expression of the marker gene by each of the two separate cellular hosts.

25. The method of claim 24, wherein one of the two cellular hosts infected in stage (f) expresses the receptor CCR5 and the other expresses the receptor CXCR4.

26. The method of claim 1, wherein said method further comprises determining the susceptibility of an HIV virus to an inhibitor compound that targets the co receptors of HIV-1, wherein said inhibitor compound that targets the co-receptors of HIV-1 is added or not added during cultivation stage (e), wherein the infection of stage (f) is carried out on two separate cellular hosts and wherein stage (g) comprises the comparison of the expression of the marker gene by each of the two separate cellular hosts.

27. The method of claim 1, wherein said method further comprises analyzing the tropism of an HIV virus for a cellular receptacle, wherein the infection of stage (f) with the viral particles that are obtained in stage (e) is carried out on two separate cellular hosts, and stage (g) comprises the comparison of the expression of the marker gene by each of the two separate cellular hosts.

28. The method of claim 1, wherein said method further comprises analyzing the susceptibility of an HIV virus to an inhibitor compound that targets the co-receptors of HIV-1, wherein said inhibitor compound that targets the co-receptors of HIV-1 is added during the cultivation of stage (d), wherein the infecting of stage (f) with the viral particles that are obtained in stage (e) is carried out on two separate cellular hosts and wherein stage (g) comprises the comparison of the expression of the marker gene by each of the two separate cellular hosts.

29. The method of claim 1, wherein said method further comprises determining the infectivity or the replicative capacity of an HIV virus, wherein stage (g) comprises the comparison of the expression of the marker gene by the second cellular host that is infected with the viral particles that are obtained by applying stages (a) to (f) to a biological sample of a patient, and the expression of the marker gene by the same second cellular host infected with reference viral particles that are obtained by applying stages (a) to (f) to a sample that contains a reference virus.

30. The method of claim 29, wherein the reference viral particles that are obtained from a reference virus are viral particles that are obtained by the application of stages (a) to (f) to a biological sample of the same patient at a previous stage of the therapeutic treatment or before the latter.

31. The method of claim 1, wherein said method further comprises determining the susceptibility of an HIV virus to hydroxyurea, wherein hydroxyurea is added or not added either during cultivation stage (e), or to the second cellular host, before the infection of the latter in stage (f) and wherein stage (g) comprises the comparison of the expression of the marker gene with and without hydroxyurea.

32. The method of claim 1, wherein the cultivating stage (e) is carried out during a period from 12 hours to 72 hours.

33. A kit for the implementation of a method according to claim 1, wherein said kit comprises:
i. A pair of primers that frame a nucleic acid sequence of the viral genome that can carry at least one mutation,
ii. a vector that comprises the parts of a genome of an HIV virus that are necessary to the viral replication except for the segment amplified with the primers that are defined by (i) and the gene that codes for the envelope protein,
iii. a second vector that comprises a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents necessary for carrying out the amplification by PCR,
vii. the products and reagents that make it possible to detect the expressed marker.

34. The kit of claim 33, wherein said kit comprises:
i. The sequence primer pairs:
SEQ ID No: 1 and SEQ ID No: 2
SEQ ID No: 3 and SEQ ID No: 4
ii. a retroviral vector that is deleted from the region of the pol reading frame that codes for the HIV-1 protease that extends from residues 1505 to 2565 inclusive, deleted from the envelope region and comprising a unique restriction site M1u1,
iii. a pseudotype virus with a gene that codes for an envelope virus,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

35. The kit of claim 33, wherein said kit comprises:
i. the sequence primer pairs:
SEQ ID No: 5 and SEQ ID No: 7
SEQ ID No: 6 and SEQ ID No: 8
ii. a retroviral vector that is deleted from the region of the pol reading frame that codes for the HIV-1 reverse transcriptase that extends from residues 2618 to 2872 inclusive and that comprises a unique restriction site M1uI,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

36. The kit of claim 33, wherein said kit comprises:
i. the sequence primer pairs:
SEQ ID No: 9 and SEQ ID No: 10
SEQ ID No: 11 and SEQ ID No: 12
ii. a retroviral vector that is deleted from the entire region of the pol reading frame that codes for the HIV-1 integrase that extends from residues 4228 to 5093 inclusive and the region that codes for the viral envelope between positions 6343 and 7611 inclusive,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

37. The kit of claim 33, wherein said kit comprises:
i. the sequence primer pairs:
SEQ ID No: 13 and SEQ ID No: 14
SEQ ID No: 15 and SEQ ID No: 16
ii. a retroviral vector that is deleted from the entire region that codes for the extracellular portion of sub-unit gp41 of the HIV-1 envelope that extends from residues 7745 to 8263 inclusive and comprises a unique restriction site Mu11,
iii. a virus that is pseudotyped by a gene that codes for an envelope protein,
iv. a first cellular host that can be infected by an HIV virus,
v. a second cellular host that can be infected by an HIV virus and that comprises a market gene that can be activated only following viral infection,
vi. the products and reagents that are necessary for carrying out the amplification by PCR,
vii. the products and reagents that make it possible to detect the expressed marker.

38. The kit of claim 33, wherein said kit comprises:
i. the sequence primer pairs:
SEQ ID No: 23 and SEQ ID No: 24
SEQ ID No: 25 and SEQ ID No: 26 with SEQ ID No: 27
i. a retroviral vector that is deleted from the entire region that codes for the extracellular portion of sub-unit gp41 of the HIV-1 envelope that extends from residues 7745 to 8263 inclusive and comprises a unique restriction site Mu1,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

39. The kit of claim 33, wherein said kitcomprises:
i. the sequence primer pairs:
SEQ ID No: 17 and SEQ ID No: 14
SEQ ID No: 18 and SEQ ID No: 16
ii. a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mu11,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only by viral particles,
vi. the products and reagents that are necessary for carrying out the amplification by PCR,
vii. the products and reagents that make it possible to detect the expressed marker.

40. The kit of claim 33, wherein said kit comprises:
i. the sequence primer pairs:
SEQ ID No: 17 and SEQ ID No: 24
SEQ ID No: 18 and SEQ ID No: 26 and SEQ ID No: 27
ii. a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mull,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only by viral particles,
vi. the products and reagents that are necessary for carrying out the amplification by PCR,
vii. the products and reagents that make it possible to detect the expressed marker.

41. The kit of claim 33, wherein said kit comprises:
i. the sequence primer pairs:
SEQ ID No: 19 and SEQ ID No: 20
SEQ ID No: 21 and SEQ ID No: 22
ii. a retroviral vector that is deleted from the region, coding for the domaims that extend from loop VI to loop V3 of the HIV-1 envelope that extends from 6617 to 7250 inclusive and that comprises a unique restriction site NheI,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

42. The kit of claim 33, wherein said kit comprises:
i. the sequence primer pairs:
(SEQ ID No: 5) and (SEQ ID No: 6),
(SEQ ID No: 7) and (SEQ ID No: 8)
ii. a retroviral vector that is deleted from the region of the pol reading frame that codes for the HIV-1 reverse transcriptase that extends from residues 2618 to 2872 inclusive and comprises a unique restriction site M1uI,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

43. The kit of claim 33, wherein said kit comprises:
i. the sequence primer pairs:
(SEQ ID No: 28) and (SEQ ID No: 29) and
(SEQ ID No: 30) and (SEQ ID No: 24)
ii. a retroviral vector that is deleted from the entire region that codes for the majority of sub-unit gp120 and the extracellular portion of the gp41 of the HIV-1 envelope that extends from residues 6480 to 8263 inclusive and comprises a unique restriction site Mull,
iii. a virus that is pseudotyped by a gene that codes for an envelope protein,
iv. a first cellular host that can be infected by an HIV virus,
v. a second cellular host that can be infected by an HIV virus and that comprises a marker gene that can be activated only following viral infection,
vi. the products and reagents that are necessary for carrying out the amplification by PCR,
vii. the products and reagents that make it possible to detect the expressed marker.

44. The method of claim 1, wherein step (c) further comprises the parts of the genome of an HIV virus that are necessary for the viral replication except for the amplified segment in stage (b) and with the exception of the gene that codes for the envelope protein.

45. The method of claim 1, wherein step (d) further comprises transfecting a first cellular host with a second vector that comprises a gene that codes for an envelope protein if the envelope gene is deleted from the vector that is prepared in stage (c).

46. The method of claim 1, wherein the viral particles of step (f) further comprise a marker gene that can be activated only following viral infection.

47. The method of claim 9, wherein said inhibitor compound of reverse transcriptase is added or not added at different concentrations to the second cellular host.

48. The method of claim 12, wherein said inhibitor compound of the integrase is added or not added at different concentrations.

49. The method of claim 16, wherein said mixture of FuD1 and FuD2 is carried out in a ratio of between (10%:90%) and (90%:10%).

50. The method of claim 49, wherein said mixture is carried out in a ration of between (60%:40%) and (40%:60%).

51. The method of claim 18, wherein said mixture of FuD1 and FuD2 is carried out in a ratio that is between (10%:90%) and (90%:10%).

52. The method of claim 51, wherein said mixture is carrided out in a ratio that is between (60%:40%) and (40%:60%).

53. The method of claim 21, wherein said fusion-inhibitor compound is added at different concentrations during the cultivation of the cellular host.

54. The method of claim 22, wherein said compound that inhibits entry is added at different concentrations to the cellular host.

55. The method of claim 23, wherein said method is carried out without antibodies and with antibodies at different concentrations.

56. The method of claim 26, wherein said inhibitor compound that targets the co-receptors of HIV-1 is added or not added at different concentrations during cultivation stage (e).

57. The method of claim 28, wherein said inhibitor compound that targets the co-receptors of HIV-1 is added at different concentrations during the cultivation of stage (d).

58. The method of claim 31, wherein the hydroxyurea is added or not added at different concentrations.

59. The method of claim 32, wherein the cultivating stage (e) is carried out during a period from 24 hours to 48 hours.


SEQUENCE LISTING (31 sequences)

SEQ ID NO: 1 - DNA, 20 bp, Artificial Sequence
  Primer
  misc_feature (1)..(20): Sequence Fit A for the amplificatioin of an area of gene that codes for the protease
tcacctagaactttaaatgc

SEQ ID NO: 2 - DNA, 23 bp, Artificial Sequence
  Primer
  misc_feature (1)..(23): Sequence Pro A for the amplificatioin of an area of gene that codes for the protease
ggcaaatactggagtattgtatg

SEQ ID NO: 3 - DNA, 20 bp, Artificial Sequence
  Primer
  misc_feature (1)..(20): Sequence Fit B for the amplificatioin of an area of gene that codes for the protease
agaactttaaatgcatgggt

SEQ ID NO: 4 - DNA, 23 bp, Artificial Sequence
  Primer
  misc_feature (1)..(23): Sequence Pro B for the amplificatioin of an area of gene that codes for the protease
ggagtattgtatggattttcagg

SEQ ID NO: 5 - DNA, 20 bp, Artificial Sequence
  Primer
  misc_feature (1)..(20): Sequence MJ33 for the amplification of an area of gene of the reverse transcriptase
agtaggacctacacctgtca

SEQ ID NO: 6 - DNA, 20 bp, Artificial Sequence
  Primer
  misc_feature (1)..(20): Sequence RT-EXT for the amplification of a region of gene of the reverse transcriptase
ttcccaatgcatattgtgag

SEQ ID NO: 7 - DNA, 32 bp, Artificial Sequence
  Primer
  misc_feature (1)..(32): Sequence A35 for the amplification of the region of gene of the reverse transcriptase
ttggttgcataaattttcccattagtcctatt

SEQ ID NO: 8 - DNA, 20 bp, Artificial Sequence
  Primer
  misc_feature (1)..(20): Sequence RT-IN for the amplification of a region of gene of the reverse transcriptase
ttcccaatgcatattgtgag

SEQ ID NO: 9 - DNA, 22 bp, Artificial Sequence
  Primer
  misc_feature (1)..(20): Sequence INT B+ for the amplification of a region of gene of the integrase
gttactaatagaggaagacaaa

SEQ ID NO: 10 - DNA, 19 bp, Artificial Sequence
  Primer
  misc_feature (1)..(19): Sequence INT B- for the amplification of a region of gene of the integrase
ttttggtgttattaatgct

SEQ ID NO: 11 - DNA, 20 bp, Artificial Sequence
  Primer
  misc_feature (1)..(20): Sequence INT V+ for the amplification of the region of the integrase
caccctaactgacacaacaa

SEQ ID NO: 12 - DNA, 20 bp, Artificial Sequence
  Primer
  misc_feature (1)..(20): Sequence INT V- for the amplification of the region of gene of the integrase
aaggcctttcttatagcaga

SEQ ID NO: 13 - DNA, 20 bp, Artificial Sequence
  Primer
  misc_feature (1)..(20): Sequence FIN-A for the amplification of the region of gene of the envelope
tcaaatattacagggctgct

SEQ ID NO: 14 - DNA, 18 bp, Artificial Sequence
  Primer
  misc_feature (1)..(18): Sequence FIN-B for the amplification of a region of gene of the envelope
tagctgaagaggcacagg

SEQ ID NO: 15 - DNA, 20 bp, Artificial Sequence
  Primer
  misc_feature (1)..(20): Sequence FIN-C for the amplification of a region of gene of the envelope
ctattaacaagagatggtgg

SEQ ID NO: 16 - DNA, 19 bp, Artificial Sequence
  Primer
  misc_feature (1)..(19): Sequence FIN-D for the amplification of a region of gene of the envelope
tccaccttcttcttcgatt

SEQ ID NO: 17 - DNA, 27 bp, Artificial Sequence
  Primer
  misc_feature (1)..(26): Sequence NEU-A for the amplification of a region of gene of the envelope
tagaaagagcagaagacagtggcaatg

SEQ ID NO: 18 - DNA, 23 bp, Artificial Sequence
  Primer
  misc_feature (1)..(23): Sequence NEU-C for the amplification of a region of gene of the envelope
gtgggtcacagtctattatgggg

SEQ ID NO: 19 - DNA, 28 bp, Artificial Sequence
  Primer
  misc_feature (1)..(28): Sequence E00 for the amplification of a region of gene of the envelope
tagaaagagcagaagacagtggcaatga

SEQ ID NO: 20 - DNA, 21 bp, Artificial Sequence
  Primer
  misc_feature (1)..(20): Sequence ES8B for the amplification of a region of gene of the envelope
cacttctccaattgtccctca

SEQ ID NO: 21 - DNA, 27 bp, Artificial Sequence
  Primer
  misc_feature (1)..(27): Sequence E20 for the amplification of a region of gene of the envelope
gggccacacatgcctgtgtacccacag

SEQ ID NO: 22 - DNA, 24 bp, Artificial Sequence
  Primer
  misc_feature (1)..(24): Sequence E115 for the amplfication of a region of gene of the envelope
agaaaaattcccctccacaattaa

SEQ ID NO: 23 - DNA, 22 bp, Artificial Sequence
  Primer
  misc_feature (1)..(22): Sequence FuA for the amplification of a region of gene of the envelope
aagcaatgtatgcccctcccat

SEQ ID NO: 24 - DNA, 23 bp, Artificial Sequence
  Primer
  misc_feature (1)..(23): Sequence FuB for the amplification of a part of gene of the envelope
ggtggtagctgaagaggcacagg

SEQ ID NO: 25 - DNA, 25 bp, Artificial Sequence
  Primer
  misc_feature (1)..(25): Sequence FuC for the amplification of a part of gene of the envelope
atatgagggacaattggagaagtga

SEQ ID NO: 26 - DNA, 26 bp, Artificial Sequence
  Primer
  misc_feature (1)..(26): Sequence FuD1 used to amplify a part of gene of the envelope
tctgtctctctctccaccttcttctt

SEQ ID NO: 27 - DNA, 26 bp, Artificial Sequence
  Primer
  misc_feature: Sequence FuD2 for the amplification of a part of gene of the envelope
tctgtcttgctctccaccttcttctt

SEQ ID NO: 28 - DNA, 30 bp, Artificial Sequence
  Primer
  misc_feature (1)..(30): Primer ED3
ttaggcatctcctatggcaggaagaagcgg

SEQ ID NO: 29 - DNA, 26 bp, Artificial Sequence
  Primer
  misc_feature (1)..(26): Primer E01 for the amplification of a part of the envelope
tccagtccccccttttcttttaaaaa

SEQ ID NO: 30 - DNA, 24 bp, Artificial Sequence
  Primer
  misc_feature (1)..(24): Primer E10 for the amplification of a part of the envelope
gtgggtcacagtctattatggggt

SEQ ID NO: 31 - DNA, 19 bp, Artificial Sequence
  Primer
  misc_feature (1)..(19): Primer gag+, allowing the amplification of a part of gag
aggggcaaatggtacatca